(19) United States
(12) Patent Application Publication
(10) Pub. No.: US 2003/0109470 A1
(43) Pub. Date: Jun. 12, 2003

(54) Nucleic acid sequences useful in the detection and treatment of various cancers

(76) Inventors: Aya Jakobovits, Beverly Hills, CA (US); Pia M. Challita-Eid, Encino, CA (US); Rene S. Hubert, Los Angeles, CA (US); Wangmao Ge, Culver City, CA (US); Daniel E.H. Afar, Brisbane, CA (US)

Correspondence Address: Kate H. Murashige, Morrison & Foerster LLP, Suite 500, 3811 Valley Centre Drive, San Diego, CA, 92130, US

(21) Appl. No.: 10/121,019

(22) Filed: Apr. 10, 2002

Related U.S. Application Data
(60) Provisional application No. 60/286,630, filed on Apr. 25, 2001.
(60) Provisional application No. 60/282,739, filed on Apr. 10, 2001.
(60) Provisional application No. 60/283,112, filed on Apr. 10, 2001.

(51) Int. Cl. A61K 48/00; C07H 21/04; C12N 15/85

(52) U.S. Cl. 514/44; 536/23.2; 435/455; 435/320.1

(57) ABSTRACT

Novel nucleic acid sequences named and set forth in FIG. 1 and FIG. 2, and variants thereof, are described wherein a nucleic acid sequence of the invention exhibits tissue specific expression in normal adult tissue, and is aberrantly expressed in the cancers such as those listed in Table I. Consequently, expression of a nucleic acid sequence of FIG. 1 or FIG. 2 provides diagnostic, prognostic, prophylactic and/or therapeutic targets for cancer.

### CROSS-REFERENCE TO RELATED APPLICATIONS

This application claims priority to U.S. Provisional Application Serial No. 60/283,112 filed Apr. 10, 2001; U.S. Provisional Application Serial No. 60/282,739, filed Apr. 10, 2001; and, U.S. Provisional Application Serial No. 60/286,630 filed Apr. 25, 2001, and U.S. Utility Application Serial No. ______ filed Apr. 10, 2002 entitled “Nucleic Acids and Corresponding Proteins Useful in the Detection and Treatment of Various Cancers” (attorney Docket No. 511582004000). The contents of each of which are hereby incorporated by reference in their entirety.

### STATEMENT OF RIGHTS TO INVENTIONS MADE UNDER FEDERALLY SPONSORED RESEARCH

Not applicable.

### FIELD OF THE INVENTION

The invention described herein relates to genes, e.g., in FIG. 1 and FIG. 2, expressed in certain cancers, and to diagnostic and therapeutic methods and compositions useful in the management of cancers that express a gene of FIG. 1 or FIG. 2.

### BACKGROUND OF THE INVENTION

Cancer is the second leading cause of human death next to coronary disease. Worldwide, millions of people die from cancer every year. In the United States alone, as reported by the American Cancer Society, cancer causes the death of well over a half-million people annually, with over 1.2 million new cases diagnosed per year. While deaths from heart disease have been declining significantly, those resulting from cancer generally are on the rise. In the early part of the next century, cancer is predicted to become the leading cause of death.

Worldwide, several cancers stand out as the leading killers. In particular, carcinomas of the lung, prostate, breast, colon, pancreas, and ovary represent the primary causes of cancer death. These and virtually all other carcinomas share a common lethal feature. With very few exceptions, metastatic disease from a carcinoma is fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients experience physical debilitations following treatment. Furthermore, many cancer patients experience a recurrence.

Worldwide, prostate cancer is the fourth most prevalent cancer in men. In North America and Northern Europe, it is by far the most common cancer in males and is the second leading cause of cancer death in men. In the United States alone, well over 30,000 men die annually of this disease—second only to lung cancer. Despite the magnitude of these figures, there is still no effective treatment for metastatic prostate cancer. Surgical prostatectomy, radiation therapy, hormone ablation therapy, surgical castration and chemotherapy continue to be the main treatment modalities. Unfortunately, these treatments are ineffective for many and are often associated with undesirable consequences.

On the diagnostic front, the lack of a prostate tumor marker that can accurately detect early-stage, localized tumors remains a significant limitation in the diagnosis and management of this disease. Although the serum prostate specific antigen (PSA) assay has been a very useful tool, however its specificity and general utility is widely regarded as lacking in several important respects.

Progress in identifying additional specific markers for prostate cancer has been improved by the generation of prostate cancer xenografts that can recapitulate different stages of the disease in mice. The LAPC (Los Angeles Prostate Cancer) xenografts are prostate cancer xenografts that have survived passage in severe combined immune deficient (SCID) mice and have exhibited the capacity to mimic the transition from androgen dependence to androgen independence (Klein et al., 1997, Nat. Med. 3:402). More recently identified prostate cancer markers include PCTA-1 (Su et al., 1996, Proc. Natl. Acad. Sci. USA 93: 7252), prostate-specific membrane (PSM) antigen (Pinto et al., Clin Cancer Res 1996 Sep 2 (9): 1445-51), STEAP (Hubert, et al., Proc Natl Acad Sci U S A. 1999 Dec. 7, 1996(25): 14523-8) and prostate stem cell antigen (PSCA) (Reiter et al., 1998, Proc. Natl. Acad. Sci. USA 95: 1735).

While previously identified markers such as PSA, PSM, PCTA and PSCA have facilitated efforts to diagnose and treat prostate cancer, there is need for the identification of additional markers and therapeutic targets for prostate and related cancers in order to further improve diagnosis and therapy.

Renal cell carcinoma (RCC) accounts for approximately 3 percent of adult malignancies. Once adenomas reach a diameter of 2 to 3 cm, malignant potential exists. In the adult, the two principal malignant renal tumors are renal cell adenocarcinoma and transitional cell carcinoma of the renal pelvis or ureter. The incidence of renal cell adenocarcinoma is estimated at more than 29,000 cases in the United States, and more than 11,600 patients died of this disease in 1998. Transitional cell carcinoma is less frequent, with an incidence of approximately 500 cases per year in the United States.

Surgery has been the primary therapy for renal cell adenocarcinoma for many decades. Until recently, metastatic disease has been refractory to any systemic therapy. With recent developments in systemic therapies, particularly immunotherapies, metastatic renal cell carcinoma may be approached aggressively in appropriate patients with a possibility of durable responses. Nevertheless, there is a remaining need for effective therapies for these patients.

Of all new cases of cancer in the United States, bladder cancer represents approximately 5 percent in men (fifth most common neoplasm) and 3 percent in women (eighth most common neoplasm). The incidence is increasing slowly, concurrent with an increasing older population. In 1998, there was an estimated 54,500 cases, including 39,500 in men and 15,000 in women. The age-adjusted incidence in the United States is 32 per 100,000 for men and 8 per 100,000 in women. The historic male/female ratio of 3:1 may be decreasing related to smoking patterns in women. There were an estimated 11,000 deaths from bladder cancer in 1998 (7,800 in men and 3,900 in women). Bladder cancer incidence and mortality strongly increase with age and will be an increasing problem as the population becomes more elderly.

Most bladder cancers recur in the bladder. Bladder cancer is managed with a combination of transurethral resection of the bladder (TUR) and intravesical chemotherapy or immunotherapy. The multifocal and recurrent nature of bladder cancer points out the limitations of TUR. Most muscle-invasive cancers are not cured by TUR alone. Radical cystectomy and urinary diversion is the most effective means to eliminate the cancer but carry an undeniable impact on urinary and sexual function. There continues to be a significant need for treatment modalities that are beneficial for bladder cancer patients.

An estimated 130,200 cases of colorectal cancer occurred in 2000 in the United States, including 93,800 cases of colon cancer and 36,400 of rectal cancer. Colorectal cancers are the third most common cancers in men and women. Incidence rates declined significantly during 1992-1996 (−2.1% per year). Research suggests that these declines have been due to increased screening and polyp removal, preventing progression of polyps to invasive cancers. There were an estimated 56,300 deaths (47,700 from colon cancer, 8,600 from rectal cancer) in 2000, accounting for about 11% of all U.S. cancer deaths.

At present, surgery is the most common form of therapy for colorectal cancer, and for cancers that have not spread, it is frequently curative. Chemotherapy, or chemotherapy plus radiation, is given before or after surgery to most patients whose cancer has deeply perforated the bowel wall or has spread to the lymph nodes. A permanent colostomy (creation of an abdominal opening for elimination of body wastes) is occasionally needed for colon cancer and is infrequently required for rectal cancer. There continues to be a need for effective diagnostic and treatment modalities for colorectal cancer.

There were an estimated 164,100 new cases of lung and bronchial cancer in 2000, accounting for 14% of all U.S. cancer diagnoses. The incidence rate of lung and bronchial cancer is declining significantly in men, from a high of 86.5 per 100,000 in 1984 to 70.0 in 1996. In the 1990s, the rate of increase among women began to slow. In 1996, the incidence rate in women was 42.3 per 100,000.

Lung and bronchial cancer caused an estimated 156,900 deaths in 2000, accounting for 28% of all cancer deaths. During 1992-1996, mortality from lung cancer declined significantly among men (−1.7% per year) while rates for women were still significantly increasing (0.9% per year). Since 1987, more women have died each year of lung cancer than breast cancer, which, for over 40 years, was the major cause of cancer death in women. Decreasing lung cancer incidence and mortality rates most likely resulted from decreased smoking rates over the previous 30 years; however, decreasing smoking patterns among women lag behind those of men. Of concern, although the declines in adult tobacco use have slowed, tobacco use in youth is increasing again.

Treatment options for lung and bronchial cancer are determined by the type and stage of the cancer and include surgery, radiation therapy, and chemotherapy. For many localized cancers, surgery is usually the treatment of choice. Because the disease has usually spread by the time it is discovered, radiation therapy and chemotherapy are often needed in combination with surgery. Chemotherapy alone or combined with radiation is the treatment of choice for small cell lung cancer; on this regimen, a large percentage of patients experience remission, which in some cases is long lasting. There is however, an ongoing need for effective treatment and diagnostic approaches for lung and bronchial cancers.

An estimated 182,800 new invasive cases of breast cancer were expected to occur among women in the United States during 2000. Additionally, about 1,400 new cases of breast cancer were expected to be diagnosed in men in 2000. After increasing about 4% per year in the 1980s, breast cancer incidence rates in women have leveled off in the 1990s to about 110.6 cases per 100,000.

In the U.S. alone, there were an estimated 41,200 deaths (40,800 women, 400 men) in 2000 due to breast cancer. Breast cancer ranks second among cancer deaths in women. According to the most recent data, mortality rates declined significantly during 1992-1996 with the largest decreases in younger women, both white and black. These decreases were probably the result of earlier detection and improved treatment.

Taking into account the medical circumstances and the patient's preferences, treatment of breast cancer may involve lumpectomy (local removal of the tumor) and removal of the lymph nodes under the arm; mastectomy (surgical removal of the breast) and removal of the lymph nodes under the arm; radiation therapy; chemotherapy; or hormone therapy. Often, two or more methods are used in combination. Numerous studies have shown that, for early stage disease, long-term survival rates after lumpectomy plus radiotherapy are similar to survival rates after modified radical mastectomy. Significant advances in reconstruction techniques provide several options for breast reconstruction after mastectomy. Recently, such reconstruction has been done at the same time as the mastectomy.

Local excision of ductal carcinoma in situ (DCIS) with adequate amounts of surrounding normal breast tissue may prevent the local recurrence of the DCIS. Radiation to the breast and/or tamoxifen may reduce the chance of DCIS occurring in the remaining breast tissue. This is important because DCIS, if left untreated, may develop into invasive breast cancer. Nevertheless, there are serious side effects or sequelae to these treatments. There is, therefore, a need for efficacious breast cancer treatments.

There were an estimated 23,100 new cases of ovarian cancer in the United States in 2000. It accounts for 4% of all cancers among women and ranks second among gynecologic cancers. During 1992-1996, ovarian cancer incidence rates were significantly declining. Consequent to ovarian cancer, there were an estimated 14,000 deaths in 2000. Ovarian cancer causes more deaths than any other cancer of the female reproductive system.

Surgery, radiation therapy, and chemotherapy are treatment options for ovarian cancer. Surgery usually includes the removal of one or both ovaries, the fallopian tubes (salpingo-oophorectomy), and the uterus (hysterectomy). In some very early tumors, only the involved ovary will be removed, especially in young women who wish to have children. In advanced disease, an attempt is made to remove all intra-abdominal disease to enhance the effect of chemotherapy. There continues to be an important need for effective treatment options for ovarian cancer.

There were an estimated 28,300 new cases of pancreatic cancer in the United States in 2000. Over the past 20 years, rates of pancreatic cancer have declined in men. Rates among women have remained approximately constant but may be beginning to decline. Pancreatic cancer caused an estimated 28,200 deaths in 2000 in the United States. Over the past 20 years, there has been a slight but significant decrease in mortality rates among men (about −0.9% per year) while rates have increased slightly among women.

Surgery, radiation therapy, and chemotherapy are treatment options for pancreatic cancer. These treatment options can extend survival and/or relieve symptoms in many patients but are not likely to produce a cure for most. There is a significant need for additional therapeutic and diagnostic options for pancreatic cancer.

### SUMMARY OF THE INVENTION

The present invention relates to genes set forth in FIG. 1 and FIG. 2, that have now been found to be over-expressed in the cancer(s) listed in Table I. Northern blot expression analysis of the genes of FIG. 1 and FIG. 2 in normal tissues shows a restricted expression pattern in adult tissues. The nucleotide sequences are provided in FIG. 1 and FIG. 2. The tissue-related expression profile of the genes set forth in FIG. 1 and FIG. 2 in normal adult tissues, combined with the over-expression observed in the tumors listed in Table I, shows that the genes of FIG. 1 and FIG. 2 are aberrantly over-expressed in certain cancers, and thus serves as a useful diagnostic, prophylactic, prognostic, and/or therapeutic target for cancers of the tissue(s) such as those listed in Table I.

The invention provides polynucleotides corresponding or complementary to all or part of the genes of FIG. 1 or FIG. 2, corresponding/related mRNAs, coding and/or complementary sequences, preferably in isolated form, including polynucleotides encoding a gene of FIG. 1 or FIG. 2-related protein and fragments of 4, 5, 6, 7, 8, 9, 10, 11, 12, 13, 14, 15, 16, 17, 18, 19, 20, 21, 22, 23, 24, 25, or more than 25 contiguous amino acids of a gene of FIG. 1 or FIG. 2-related protein; at least 30, 35, 40, 45, 50, 55, 60, 65, 70, 80, 85, 90, 95, 100 or more than 100 contiguous amino acids of a gene of FIG. 1 or FIG. 2-related protein, as well as the peptides/proteins themselves; DNA, RNA, DNA/RNA hybrids, and related molecules such as, polynucleotides or oligonucleotides complementary or having at least a 90% homology to the genes set forth in FIG. 1 or FIG. 2 or mRNA sequences or parts thereof, and polynucleotides or oligonucleotides that hybridize to the genes set forth in FIG. 1 or FIG. 2, mRNAs, or to polynucleotides that encode a gene of FIG. 1 or FIG. 2-related protein of analogs or variants thereof; or to polynucleotides that encode fragments of a gene of FIG. 1 or FIG. 2-related protein such as any 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540,545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1025, 1050, 1075, 1100, 1125, 1150, 1175, 1200, etc., or more contiguous amino acids of a gene of FIG. 1 or FIG. 2-related protein, or an analog or variant thereof.

Also provided are means for isolating cDNAs and the genes of the invention. Recombinant DNA molecules containing genes of FIG. 1 or FIG. 2 polynucleotides, cells transformed or transduced with such molecules, and host-vector systems for the expression of the genes set forth in FIG. 1 or FIG. 2 products are also provided. The invention further provides antibodies that bind to a gene of FIG. 1 or FIG. 2-related protein and polypeptide fragments thereof, including polyclonal and monoclonal antibodies, murine and other manmalian antibodies, chimeric antibodies, humanized and fully human antibodies, and antibodies labeled with a detectable marker or therapeutic agent. In certain embodiments there is a proviso that the entire nucleic acid sequence of a gene of FIG. 1 or FIG. 2 is not encoded. In certain embodiments, the entire nucleic acid sequence of the genes of FIG. 1 or FIG. 2 is encoded, which can be in a human unit dose form.

The invention further provides methods for detecting the presence and status of FIG. 1 or FIG. 2 polynucleotides and proteins in various biological samples, as well as methods for identifying cells that express the genes set forth in FIG. 1 or FIG. 2. A typical embodiment of this invention provides methods for monitoring the gene of FIG. 1 or FIG. 2 expression in a tissue or hematology sample having or suspected of having some form of growth dysregulation such as cancer.

The invention further provides various immunogenic or therapeutic compositions and strategies for treating cancers that express a gene set forth in FIG. 1 or FIG. 2 such as cancers of tissues listed in Table I, including therapies aimed at inhibiting the transcription, translation, processing or function of the genes of FIG. 1 or FIG. 2 as well as cancer vaccines. In one aspect, the invention provides compositions, and methods comprising them, for treating a cancer that expresses a gene set forth in FIG. 1 or FIG. 2 in a human subject wherein the composition comprises a carrier suitable for human use and a human unit dose of one or more than one agent that inhibits the production or function of a gene of FIG. 1 or FIG. 2. Preferably, the carrier is uniquely for use in humans. In another aspect of the invention, the agent is a moiety that is immunoreactive with a gene of FIG. 1 or FIG. 2-related protein. Non-limiting examples of such moieties include, but are not limited to, antibodies (such as single chain, monoclonal, polyclonal, humanized, chimeric, or human antibodies), functional equivalents thereof (whether naturally occurring or synthetic), and combinations thereof. The antibodies can be conjugated to a diagnostic or therapeutic moiety. In another aspect, the agent is a small molecule as defined herein.

In another aspect, the agent comprises one or more than one peptide which comprises a cytotoxic T lymphocyte (CTL) epitope that binds an HLA class I molecule in a human to elicit a CTL response to a gene of FIG. 1 or FIG. 2-related protein and/or one or more than one peptide which comprises a helper T lymphocyte (HTL) epitope which binds an HLA class II molecule in a human to elicit an HTL response. The peptides of the invention may be on the same or on one or more separate polypeptide molecules. In a further aspect of the invention, the agent comprises one or more than one nucleic acid molecule that expresses one or more than one of the CTL or HTL response stimulating peptides as described above. In yet another aspect of the invention, the one or more than one nucleic acid molecule may express a moiety that is immunologically reactive with a gene of FIG. 1 or FIG. 2-related protein as described above. The one or more than one nucleic acid molecule may also be, or encodes, a molecule that inhibits production of a gene of FIG. 1 or FIG. 2-related protein. Non-limiting examples of such molecules include, but are not limited to, those complementary to a nucleotide sequence essential for production of a gene of FIG. 1 or FIG. 2-related protein (e.g. antisense sequences or molecules that form a triple helix with a nucleotide double helix essential for production of a gene of FIG. 1 or FIG. 2-related protein, or a ribozyme effective to lyse mRNA (sense or antisense) encoded by a gene of FIG. 1 or FIG. 2.

### BRIEF DESCRIPTION OF THE FIGURES

FIG. 1. The SSH sequences of the invention, also referred to as genes of the invention.

FIG. 2. Extended SSH sequences of FIG. 1, also referred to as genes of the invention.

FIG. 3. Expression of 105P1B7 by RT-PCR. (A) First strand cDNA was prepared from normal brain, normal prostate, LAPC-4AD, LAPC-4AD at 3 and 28 days after castration, LAPC-4AI, and Hela cancer cell lines. Normalization was performed by PCR using primers to actin and GAPDH. Semi-quantitative PCR, using primers to 105P1B7, was performed at 26 and 35 cycles of amplification. Results show expression of 105P1B7 in normal prostate and in the LAPC prostate cancer xenografts, but not in normal brain nor in the HeLa cell line. (B) First strand cDNA was prepared from vital pool 1 (liver, lung and kidney), vital pool 2 (pancreas, colon and stomach), prostate metastasis to lymph node (LN), prostate cancer pool, bladder cancer pool kidney cancer pool, colon cancer pool, lung cancer pool, ovary cancer pool, breast cancer pool, and pancreas cancer pool. Expression of 105P1B7 was detected in all cancer pools tested and in the vital pools.

FIG. 4. Expression of 105P1B7 in normal tissues. Two multiple tissue northern blots (Clontech) both with 2 μg of mRNA/lane, were probed with the 105P1B7 SSH fragment. Size standards in kilobases (kb) are indicated on the side. Results show expression of approximately 6.5 kb 105P1B7 transcript in ovary and weakly in normal prostate, but not in the other normal tissues tested.

FIG. 5. Expression of 105P1B7 in prostate cancer xenografts. RNA was extracted from normal prostate (NP), LAPC prostate cancer xenografts, LAPC-4AD, LAPC4AI, LAPC-9AD and LAPC-9AI. Northern blot with 10 μg of total RNA/lane was probed with 105P1B7 SSH sequence. Size standards in kilobases (kb) are indicated on the side. The results show strong expression of 105P1B7 in all xenografts tissues and in normal prostate.

FIG. 6. Expression of 105P1B7 in prostate cancer patient specimens. RNA was extracted from normal prostate (NP), prostate cancer patient tumors (T) and their normal adjacent tissues (N). Northern blot with 10 μg of total RNA/lane was probed with 105P1B7 SSH sequence. Size standards in kilobases (kb) are indicated on the side. The results show strong expression of 105P1B7 in normal prostate and in patient prostate cancer specimens.

FIG. 7. Expression of 152P1A2B by RT-PCR. First strand cDNA was prepared from vital pool 1 (liver, lung and kidney), vital pool 2 (pancreas, colon and stomach), LAPC prostate cancer xenograft pool (LAPC-4AD, LAPC-4AI, LAPC-9AD and LAPC-9AI), prostate cancer pool, and kidney cancer pool. Normalization was performed by PCR using primers to actin and GAPDH. Semi-quantitative PCR, using primers to 152P1A2B, was performed at 26 and 30 cycles of amplification. Results show strong expression of 83P4B8 in xenograft pool, prostate cancer pool, and kidney cancer pool. Expression was detected in the vital pool 1 but not in vital pool 2.

FIG. 8. Expression of 154P2G7 by RT-PCR. First strand cDNA was prepared from vital pool 1 (liver, lung and kidney), vital pool 2 (pancreas, colon and stomach), bladder cancer pool, kidney cancer pool, lung cancer pool, ovary cancer pool, and cancer metastasis pool. Normalization was performed by PCR using primers to actin and GAPDH. Semi-quantitative PCR, using primers to 154P2G7, was performed at 26 and 30 cycles of amplification. Results show strong expression of 154P2G7 in bladder cancer pool. Expression was also detected in kidney cancer pool, lung cancer pool, ovary cancer pool and cancer metastasis pool but not in the 2 vital pools tested.

FIG. 9. Expression of 154P2G7 in normal tissues. Two multiple tissue northern blots (Clontech), both with 2 μg of mRNA/lane, were probed with the 154P2G7 SSH fragment. Size standards in kilobases (kb) are indicated on the side. Results show expression of an approximately 1.8 kb 154P 2G7 transcript in testis. Very low expression was also detected in skeletal muscle and brain, but not in the other normal tissues tested.

FIG. 10. Expression of 154P2G7 in bladder cancer patient specimens. RNA was extracted from bladder cancer cell lines (CL; UM-UC-3, SCaBER), normal bladder (Nb), and bladder cancer patient tumors (T). Northern blots with 10 μg of total RNA were probed with the 154P2G7 SSH sequence. Size standards in kilobases are indicated on the side. Results show expression of 154P2G7 in patient bladder cancer tissues, but not in normal bladder, nor in the bladder cancer cell lines tested.

FIG. 11. Expression of 156P3A6 by RT-PCR. First strand cDNA was prepared from vital pool 1 (VP 1: liver, lung and kidney), vital pool 2 (VP2, pancreas, colon and stomach), prostate metastasis to lymph node (LN), prostate cancer pool, bladder cancer pool, kidney cancer pool, colon cancer pool, lung cancer pool, ovary cancer pool, breast cancer pool, cancer metastasis pool, and pancreas cancer pool. Normalization was performed by PCR using primers to actin and GAPDH. Semi-quantitative PCR, using primers to 156P 3A6, was performed at 26 and 30 cycles of amplification. Results show strong expression of 156P3A 6 in prostate cancer pool, colon cancer pool, and cancer metastasis pool. Expression was also detected in the other cancer pools tested and in the vital pools.

FIG. 12. Expression of 156P3A6 in normal tissues. Multiple tissue northern blot, with 10 μg of total RNA/lane, was probed with the 156P3A6 SSH fragment. Size standards in kilobases (kb) are indicated on the side. The results show exclusive expression of an approximately 3.0 kb 156P3A 6 transcript in kidney and prostate.

FIG. 13. Expression of 156P3A6 in kidney cancer patient specimens. RNA was extracted from normal kidney (Nk), kidney tumors (T) and their normal adjacent tissues (N) derived from kidney cancer patients. Northern blots with 10 μg of total RNA/lane were probed with the 156P3A6 SSH fragment. Size standards in kilobases (kb) are indicated on the side. The results show expression of 156P3A6 in kidney tumors and their normal adjacent tissues. Expression detected in kidney tumors is stronger than expression detected in normal kidney.

FIG. 14. Expression of 158P3H2B by RT-PCR. First strand cDNA was prepared (A) from vital pool 1 (VP1: liver, lung and kidney), vital pool 2 (VP2, pancreas, spleen and stomach), LAPC xenograft pool (LAPC-4AD, LAPC-4AI, LAPC-9AD and LAPC-9AI), normal prostate, bladder cancer pool, and kidney cancer pool; (B) from vital pool 1 (VP1: liver, lung and kidney), vital pool 2 (VP2, pancreas, spleen and stomach), bladder cancer pool, kidney cancer pool, colon cancer pool and lung cancer pool. Normalization was performed by PCR using primers to actin and GAPDH. Semi-quantitative PCR, using primers to 158P3H 2B, was performed at 30 cycles of amplification. Results show expression of 158P3H2B in bladder cancer pool, kidney cancer pool, colon cancer pool, and lung cancer pool but not in the normal tissues tested.

FIG. 15. Expression of 158P3H2B in normal human tissues. Two multiple tissue northern blots (Clontech) both with 2 μg of mRNA/lane, were probed with the 158P3H 2B SSH fragment. Size standards in kilobases (kb) are indicated on the side. The results show exclusive expression of a 2.4 kb 158P3H2B transcript in testis but not in the other tissues tested.

FIG. 16. Expression of 158P3H2B in bladder cancer patient samples. RNA was extracted from bladder cancer cell lines (CL: UM-UC-3, J82, SCaBER), normal bladder (Nb), bladder tumors (T) and their normal adjacent tissues (N) harvested from bladder cancer patients. Northern blots with 10 μg of total RNA/lane were probed with the 158P3H2B SSH fragment. Size standards in kilobases (kb) are indicated on the side. The results show strong expression of 158P3H2B in all 5 bladder tumors tested and in one normal adjacent tissue, but not in normal bladder. Also strong expression was seen in the two cell lines, UM-UC-3 and SCABER, and to much lower level in J82.

FIG. 17. Expression of 187P4F11 by RT-PCR. First strand cDNA was prepared from vital pool 1 (liver, lung and kidney), vital pool 2 (pancreas, colon and stomach), prostate metastasis to lymph node (LN), prostate cancer pool, breast cancer pool, and cancer metastasis pool. Normalization was performed by PCR using primers to actin and GAPDH. Semi-quantitative PCR, using primers to 187P4F11, was performed at 26 and 30 cycles of amplification. Results show strong expression of 187P4F11 in prostate cancer pool, breast cancer pool, and cancer metastasis pool, but not in the vital pool. Expression of 187P4F11 was also detected in prostate metastasis to LN indicating the 187P4F11 can be a marker for cancer metastasis.

FIG. 18. Expression of 187P4F11 in normal tissues. Two multiple tissue northern blots (Clontech), both with 2 μg of mRNA/lane, were probed with the 187P4F11 SSH fragment. Size standards in kilobases (kb) are indicated on the side. Results show absence of 187P4F11 in all 16 normal tissues tested.

FIG. 19. Expression of 187P4F11 in patient cancer specimens and normal tissues. RNA was extracted from a pool of three prostate cancers (PC), kidney cancers, as well as from normal prostate (NP), normal bladder (NB), normal kidney (NK), and normal colon (NC). Northern blot with 10 μg of total RNA/lane was probed with 187P4F11 SSH sequence. Size standards in kilobases (kb) are indicated on the side. Results show expression of 187P4F11 in the bladder cancers and kidney cancers, but not in the normal tissues tested.

FIG. 20. Expression of 187P4F11 in prostate cancer patient specimens. RNA was extracted from LAPC xenograft tissues, LAPC4AD, LAPC-4AI, LAPC-9AD, LAPC-9AI, normal prostate (NP), prostate cancer patient tumors (T) and their normal adjacent tissues (N). Northern blot with 10 μg of total RNA/lane was probed with 83P4B8 SSH sequence. Size standards in kilobases (kb) are indicated on the side. The results show strong expression of approximately 2 and 2.8 kb 187P4F11 transcripts in the patient prostate cancer specimens, but not in normal prostate, nor in the xenograft tissues.

### DETAILED DESCRIPTION OF THE INVENTION

I.) Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in Sambrook et al, Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

The terms “advanced prostate cancer”, “locally advanced prostate cancer”, “advanced disease” and “locally advanced disease” mean prostate cancers that have extended through the prostate capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined). prostate cancer. Locally advanced disease is clinically identified by palpable evidence of induration beyond the lateral border of the prostate, or asymmetry or induration above the prostate base. Locally advanced prostate. cancer is presently diagnosed pathologically following radical prostatectomy if the tumor invades or penetrates the prostatic capsule, extends into the surgical margin, or invades the seminal vesicles.

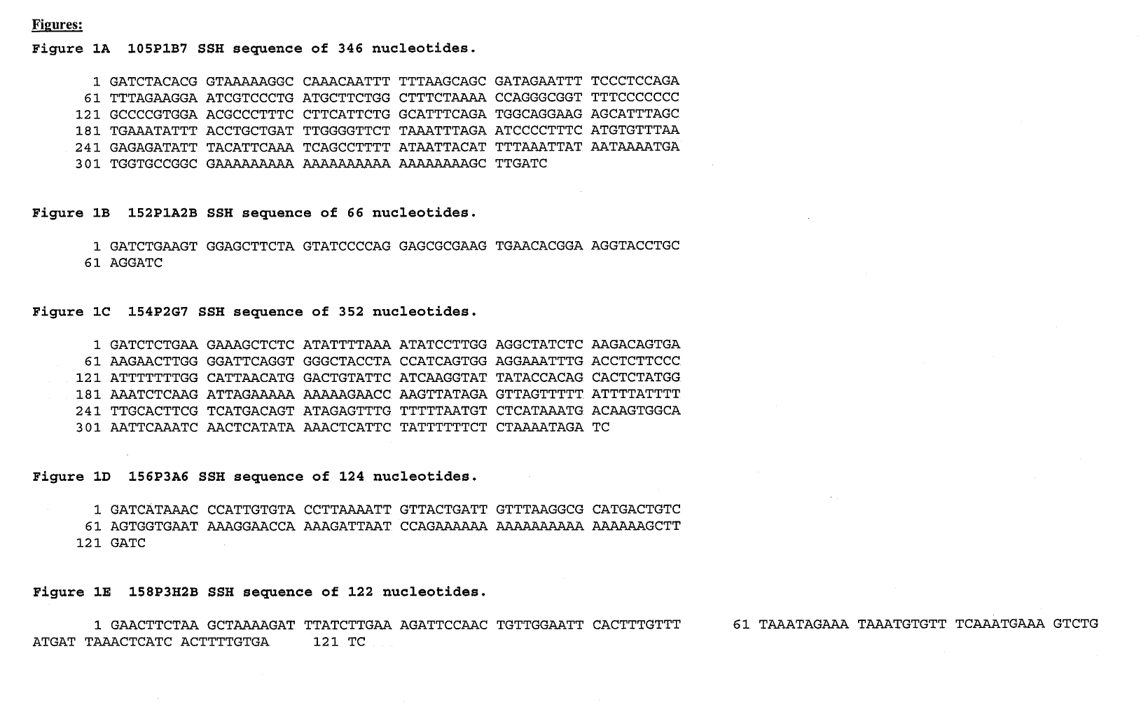

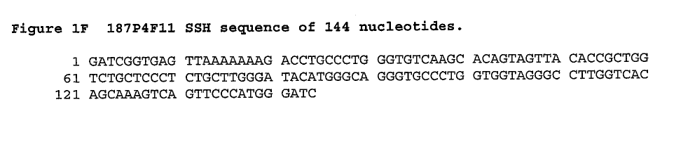

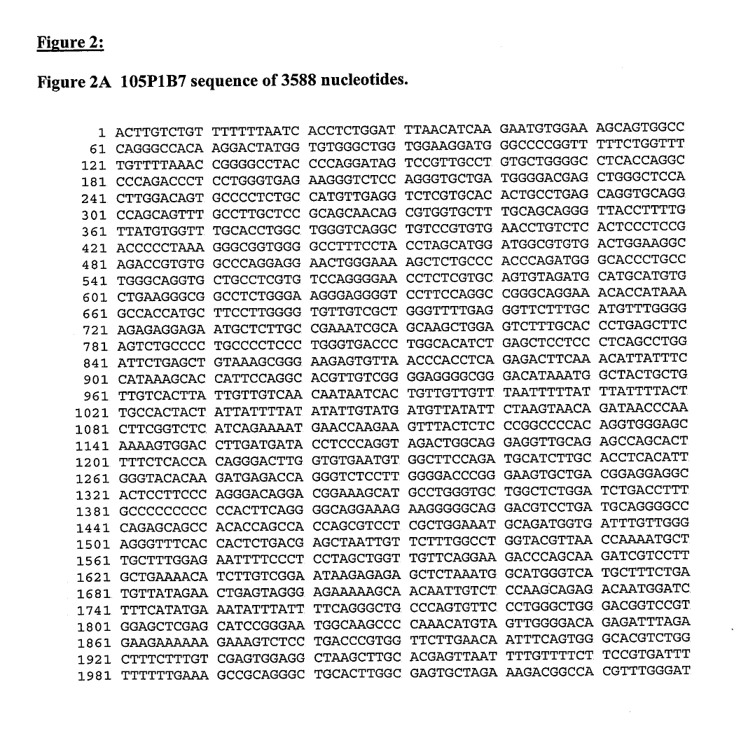

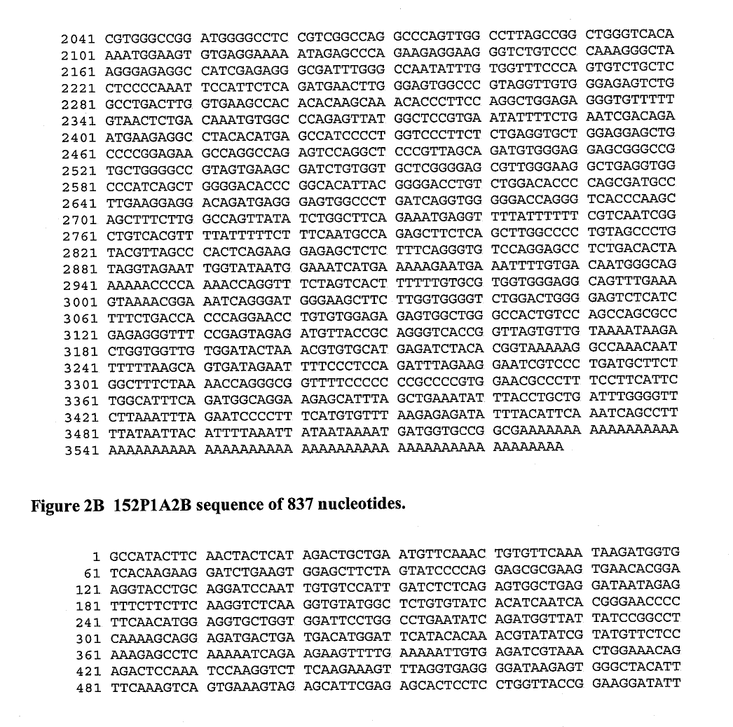

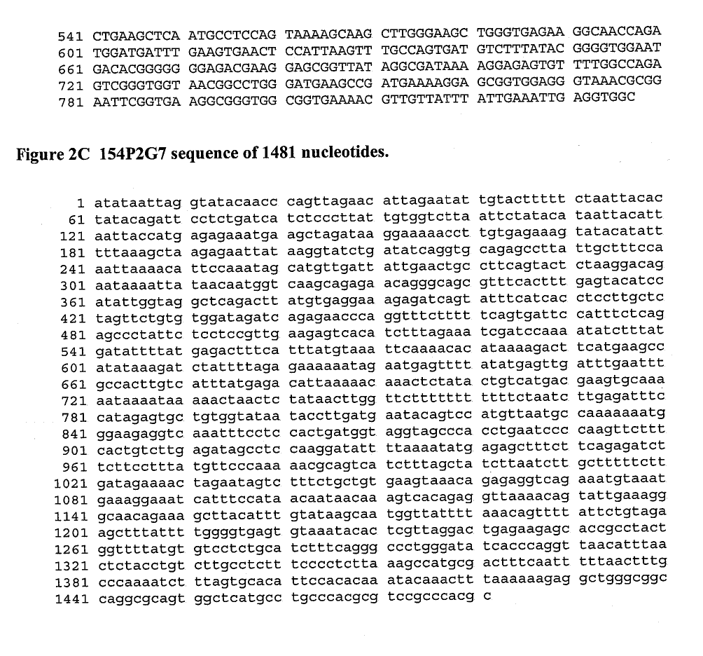

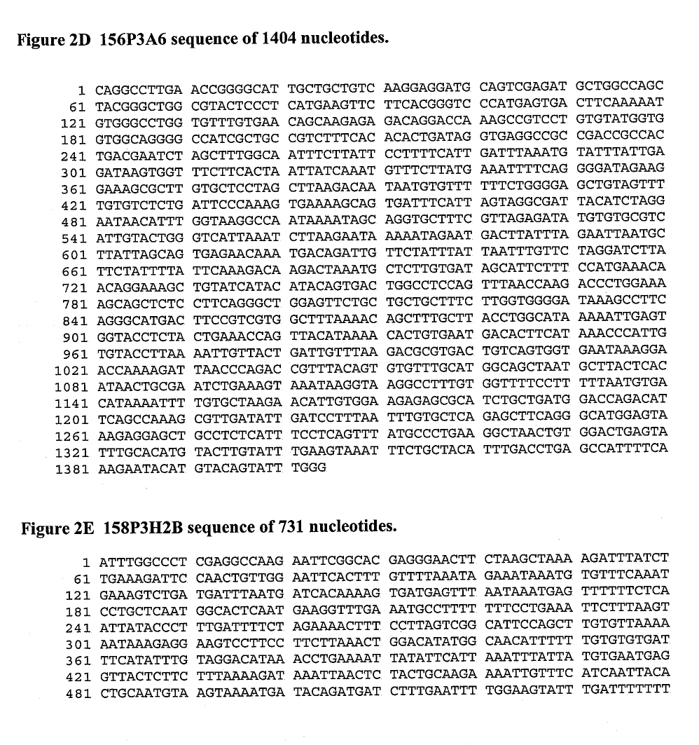

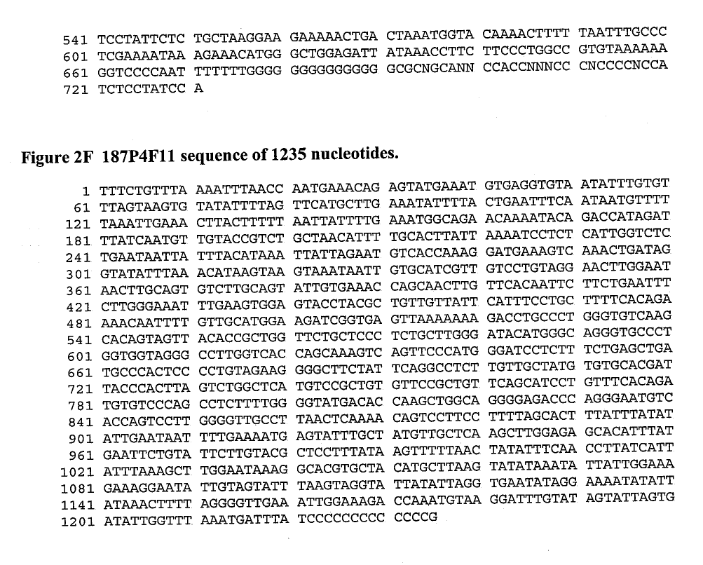

“Altering the native glycosylation pattern” is intended for purposes herein to mean deleting one or more carbohydrate moieties found in native sequence of the genes set forth in FIG. 1 or FIG. 2 (either by removing the underlying glycosylation site or by deleting the glycosylation by chemical and/or enzymatic means),. and/or adding one or more glycosylation sites that are not present in the native sequence of a native protein. In addition, the phrase includes qualitative changes in the glycosylation of the native proteins, involving a change in the nature and proportions of the various carbohydrate moieties present.

The term “analog” refers to a molecule which is structurally similar or shares similar or corresponding attributes with another molecule (e.g. a gene of FIG. 1 or FIG. 2-related protein). For example an analog of a gene of FIG. 1 or FIG. 2-related protein can be specifically bound by an antibody or T cell that specifically binds to the respective gene of FIG. 1 or FIG. 2-related protein.

The term “antibody” is used in the broadest sense. Therefore an “antibody” can be naturally occurring or man-made such as monoclonal antibodies produced by conventional hybridoma technology. Antibodies of the invention comprise monoclonal and polyclonal antibodies as well as fragments containing the antigen-binding domain and/or one or more complementarity determining regions of these antibodies that specifically bind a gene of FIG. 1 or FIG. 2-related protein.

An “antibody fragment” is defined as at least a portion of the variable region of the immunoglobulin molecule that binds to its target, i.e., the antigen-binding region. In one embodiment it specifically covers single antibodies and clones thereof (including agonist, antagonist and neutralizing antibodies) and antibody compositions with polyepitopic specificity.

The term “codon optimized sequences” refers to nucleotide sequences that have been optimized for a particular host species by replacing any codons having a usage frequency of less than about 20%. Nucleotide sequences that have been optimized for expression in a given host species by elimination of spurious polyadenylation sequences, elimination of exon/intron splicing signals, elimination of transposon-like repeats and/or optimization of GC content in addition to codon optimization are referred to herein as an “expression enhanced sequences.”

The term “cytotoxic agent” refers to a substance that inhibits or prevents the expression activity of cells, function of cells and/or causes destruction of cells. The term is intended to include radioactive isotopes chemotherapeutic agents, and toxins such as small molecule toxins or enzymatically active toxins of bacterial, fungal, plant or animal origin, including fragments and/or variants thereof. Examples of cytotoxic agents include, but are not limited to maytansinoids, yttrium, bismuth, ricin, ricin A-chain, doxorubicin, daunorubicin, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹², P³² and radioactive isotopes of Lu. Antibodies may also be conjugated to an anti-cancer pro-drug activating enzyme capable of converting the pro-drug to its active form.

The term “homolog” refers to a molecule which exhibits homology to another molecule, by for example, having sequences of chemical residues that are the same or similar at corresponding positions.

“Human Leukocyte Antigen” or “HLA” is a human class I or class II Major Histocompatibility Complex (MHC) protein (see, e.g., Stites, et al., IMMUNOLOGY, 8^{TH} ED., Lange Publishing, Los Altos, Calif. (1994).

The terms “hybridize”, “hybridizing”, “hybridizes” and the like, used in the context of polynucleotides, are meant to refer to conventional hybridization conditions, preferably such as hybridization in 50% formnamide/6XSSC/0.1% SDS/100 μg/ml ssDNA, in which temperatures for hybridization are above 37 degrees C and temperatures for washing in 0.1XSSC/0.1% SDS are above 55 degrees C.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment. For example, a polynucleotide is said to be “isolated” when it is substantially separated from contaminant polynucleotides that correspond or are complementary to genes other than the genes of FIG. 1 or FIG. 2. A skilled artisan can readily employ nucleic acid isolation procedures to obtain an isolated polynucleotide. A protein is said to be “isolated,” for example, when physical, mechanical or chemical methods are employed to remove a gene of FIG. 1 or FIG. 2-related protein from cellular constituents that are normally associated with the protein. A skilled artisan can readily employ standard purification methods to obtain an isolated gene of FIG. 1 or FIG. 2-related protein. Alternatively, an isolated protein can be prepared by chemical means.

The term “mammal” refers to any organism classified as a mammal, including mice, rats, rabbits, dogs, cats, cows, horses and humans. In one embodiment of the invention, the mammal is a mouse. In another embodiment of the invention, the mammal is a human.

The terms “metastatic prostate cancer” and “metastatic disease” mean prostate cancers that have spread to regional lymph nodes or to distant sites, and are meant to include stage D disease under the AUA system and stage T×N×M+ under the TNM system. As is the case with locally advanced prostate cancer, surgery is generally not indicated for patients with metastatic disease, and hormonal (androgen ablation) therapy is a preferred treatment modality. Patients with metastatic prostate cancer eventually develop an androgen-refractory state within 12 to 18 months of treatment initiation. Approximately half of these androgen-refractory patients die within 6 months after developing that status. The most common site for prostate cancer metastasis is bone. Prostate cancer bone metastases are often osteoblastic rather than osteolytic (i.e., resulting in net bone formation). Bone metastases are found most frequently in the spine, followed by the femur, pelvis, rib cage, skull and humerus. Other common sites for metastasis include lymph nodes, lung, liver and brain. Metastatic prostate cancer is typically diagnosed by open or laparoscopic pelvic lymphadenectomy, whole body radionuclide scans, skeletal radiography, and/or bone lesion biopsy.

The term “monoclonal antibody” refers to an antibody obtained from a population of substantially homogeneous antibodies, i.e., the antibodies comprising the population are identical except for possible naturally occurring mutations that are present in minor amounts.

A “motif”, as in biological motif of a gene of FIG. 1 or FIG. 2-related protein, refers to any pattern of amino acids forming part of the primary sequence of a protein, that is associated with a particular function (e.g. protein-protein interaction, protein-DNA interaction, etc) or modification (e.g. that is phosphorylated, glycosylated or amidated), or localization (e.g. secretory sequence, nuclear localization sequence, etc.) or a sequence that is correlated with being immunogenic, either humorally or cellularly. A motif can be either contiguous or capable of being aligned to certain positions that are generally correlated with a certain function or property. In the context of HLA motifs, “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs for HLA binding are typically different for each protein encoded by each human HLA allele and differ in the pattern of the primary and secondary anchor residues.

A “pharmaceutical excipient” comprises a material such as an adjuvant, a carrier, pH-adjusting and buffering agents, tonicity adjusting agents, wetting agents, preservative, and the like.

“Pharmaceutically acceptable” refers to a non-toxic, inert, and/or composition that is physiologically compatible with humans or other mammals.

The term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. A polynucleotide can comprise a nucleotide sequence disclosed herein wherein thymine (T), as shown for example in FIG. 1 or FIG. 2, can also be uracil (U); this definition pertains to the differences between the chemical structures of DNA and RNA, in particular the observation that one of the four major bases in RNA is uracil (U) instead of thymine (T).

The term “polypeptide” means a polymer of at least about 4, 5, 6, 7, or 8 amino acids. Throughout the specification, standard three letter or single letter designations for amino acids are used. In the art, this term is often used interchangeably with “peptide” or “protein”.

An HLA “primary anchor residue” is an amino acid at a specific position along a peptide sequence which is understood to provide a contact point between the immunogenic peptide and the HLA molecule. One to three, usually two, primary anchor residues within a peptide of defined length generally defines a “motif” for an immunogenic peptide. These residues are understood to fit in close contact with peptide binding groove of an HLA molecule, with their side chains buried in specific pockets of the binding groove. In one embodiment, for example, the primary anchor residues for an HLA class I molecule are located at position 2 (from the amino terminal position) and at the carboxyl terminal position of a 8, 9, 10, 11, or 12 residue peptide epitope in accordance with the invention. In another embodiment, for example, the primary anchor residues of a peptide that will bind an HLA class II molecule are spaced relative to each other, rather than to the termini of a peptide, where the peptide is generally of at least 9 amino acids in length.. Such analogs are used to modulate the binding affinity and/or population coverage of a peptide comprising a particular HLA motif or supermotif.

A “recombinant” DNA or RNA molecule is a DNA or RNA molecule that has been subjected to molecular manipulation in vitro.

Non-limiting examples of small molecules include compounds that bind or interact with the gene of FIG. 1 or FIG. 2-related proteins, ligands including hormones, neuropeptides, chemokines, odorants, phospholipids, and functional equivalents thereof that bind and preferably inhibit function of a gene of FIG. 1 or FIG. 2-related protein. Such non-limiting small molecules preferably have a molecular weight of less than about 10 kDa, more preferably below about 9, about 8, about 7, about 6, about 5 or about 4 kDa. In certain embodiments, small molecules physically associate with, or bind, a gene of FIG. 1 or FIG. 2-related protein; and are not found in naturally occurring metabolic pathways; and/or are more soluble in aqueous than non-aqueous solutions

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers, (1995).

“Stringent conditions” or “high stringency conditions”, as defined herein, are identified by, but not limited to, those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC (sodium chloride/sodium. citrate) and 50% formamide at 55° C., followed by a high-stringency wash consisting of 0.1× SSC containing EDTA at 55° C. “Moderately stringent conditions” are described by, but not limited to, those in Sambrook et al, Molecular Cloning: A Laboratory Manual, New York: Cold Spring Harbor Press, 1989, and include the use of washing solution and hybridization conditions (e.g., temperature, ionic strength and %SDS) less stringent than those described above. An example of moderately stringent conditions is overnight incubation at 37° C. in a solution comprising: 20% formamide, 5× SSC (150 mM NaCl, 15 mM trisodium citrate), 50 mM sodium phosphate (pH 7.6), 5× Denhardt's solution, 10% dextran sulfate, and 20 mg/mL denatured sheared salmon sperm DNA, followed by washing the filters in 1× SSC at about 37-50° C. The skilled artisan will recognize how to adjust the temperature, ionic strength, etc. as necessary to accommodate factors such as probe length and the like.

An HLA “supermotif” is a peptide binding specificity shared by HLA molecules encoded by two or more HLA alleles.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; full eradication of disease is not required.

A “transgenic animal” (e.g., a mouse or rat) is an animal having cells that contain a transgene, which transgene was introduced into the animal or an ancestor of the animal at a prenatal, e.g., an embryonic stage. A “transgene” is a DNA that is integrated into the genome of a cell from which a transgenic animal develops.

As used herein, an HLA or cellular immune response “vaccine” is a composition that contains or encodes one or more peptides of the invention. There are numerous embodiments of such vaccines, such as a cocktail of one or more individual peptides; one or more peptides of the invention comprised by a polyepitopic peptide; or nucleic acids that encode such individual peptides or polypeptides, e.g., a minigene that encodes a polyepitopic peptide. The “one or more peptides” can include any whole unit integer from 1-150 or more, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, or 150 or more peptides of the invention. The peptides or polypeptides can optionally be modified, such as by lipidation, addition of targeting or other sequences. HLA class I peptides of the invention can be admixed with, or linked to, HLA class II peptides, to facilitate activation of both cytotoxic T lymphocytes and helper T lymphocytes. HLA vaccines can also comprise peptide-pulsed antigen presenting cells, e.g., dendritic cells.

The term “variant” refers to a molecule that exhibits a variation from a described type or norm, such as a protein that has one or more different amino acid residues in the corresponding position(s) of a specifically described protein, e.g. a gene of FIG. 1 or FIG. 2-related protein. An analog is an example of a variant protein. Splice isoforms and single nucleotides polymorphisms (SNPs) are further examples of variants.

The “genes of FIG. 1 or FIG. 2-related proteins” of the invention include those specifically identified herein, as well as allelic variants, conservative substitution variants, analogs and homologs that can be isolated/generated and characterized without undue experimentation following the methods outlined herein or readily available in the art. Fusion proteins that combine parts of different gene of FIG. 1 or FIG. 2-related proteins or fragments thereof, as well as fusion proteins of a gene of FIG. 1 or FIG. 2-related protein and a heterologous polypeptide are also included. Such gene of FIG. 1 or FIG. 2-related proteins are collectively referred to as the gene of FIG. 1 or FIG. 2-related proteins, the proteins of the invention or Proteins of FIG. 1 or FIG. 2.. The term “gene of FIG. 1 or FIG. 2-related protein” refers to a polypeptide fragment or a gene of FIG. 1 or FIG. 2-related protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100 or more than 100 amino acids. In certain cases the phrase “corresponding to” or “respective” is used instead of the term “-related.”

Polynucleotides of the Invention

One aspect of the invention provides polynucleotides corresponding or complementary to all or part of a gene of FIG. 1 or FIG. 2; gene of FIG. 1 or FIG. 2-related mRNA, a coding sequence of a gene of FIG. 1 or FIG. 2, an open reading frame of a gene of FIG. 1 or FIG. 2, each of the foregoing preferably in isolated form. Polynucleotides of the invention include polynucleotides encoding a gene of FIG. 1 or FIG. 2-related proteins and fragments thereof, DNA, RNA, DNA/RNA hybrid, and related molecules, polynucleotides or oligonucleotides complementary to a FIG. 1 or FIG. 2 gene or mRNA sequence or a part thereof, and polynucleotides or oligonucleotides that hybridize to a FIG. 1 or FIG. 2 gene, mRNA, or to a FIG. 1 or FIG. 2 encoding polynucleotide (collectively, “FIG. 1 or FIG. 2 polynucleotides”). In all instances when referred to in this section, T can also be U in FIG. 1. or FIG. 2.

Embodiments of a FIG. 1 or FIG. 2 polynucleotide include: a FIG. 1 or FIG. 2 polynucleotide having the sequence shown in FIG. 1 or FIG. 2, the nucleotide sequence of the genes of FIG. 1 or FIG. 2 as shown in FIG. 1 or FIG. 2 wherein T is U; at least 10 contiguous nucleotides of a polynucleotide having the sequence as shown in FIG. 1 or FIG. 2; or, at least 10 contiguous nucleotides of a polynucleotide having the sequence as shown in FIG. 1 or FIG. 2 where T is U. For example, embodiments of the FIG. 1 or FIG. 2 nucleotides comprise, without limitation:

(1) a polynucleotide comprising, consisting essentially of, or consisting of a sequence as shown in FIG. 1 or FIG. 2 (SEQ ID NO:______ ), wherein T can also be U;

(2) a polynucleotide comprising, consisting essentially of, or consisting of a sequence as shown in FIG. 1 or FIG. 2 (SEQ ID NOs:______ ), wherein T can also be U;

(3) a polynucleotide that encodes a gene of FIG. 1 or FIG. 2-related protein that is at least 90% homologous to an entire amino acid sequence shown in FIG. 1 or FIG. 2 (SEQ ID NO:______ );

(4) a polynucleotide that encodes a gene of FIG. 1 or FIG. 2-related protein that is at least 90% identical to an entire nucleic acid sequence shown in FIG. 1 or FIG. 2;

(5) a polynucleotide that is fully complementary to a polynucleotide of any one of (1)-(4);

(6) a polynucleotide that selectively hybridizes under stringent conditions to a polynucleotide of (1) to (5);

(7) a peptide that is encoded by any of (1)-(4); and,

(8) a polynucleotide of any of (1)-(6)or peptide of (7) together with a pharmaceutical excipient and/or in a human unit dose form.

As used herein, a range is understood to specifically disclose all whole unit positions, i.e., integer positions, thereof.

Typical embodiments of the invention disclosed herein include gene of FIG. 1 or FIG. 2-related proteins, polynucleotides that encode specific portions of a gene of FIG. 1 or FIG. 2-related mRNA sequences (and those which are complementary to such sequences) such as those that encode the proteins and/or fragments thereof, for example:

4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200,205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670,675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1025, 1050, 1075, 1100, 1125, 1150, 1175, 1200, etc., or more contiguous amino acids of a peptide of the invention.

Uses Polynucleotides of the Invention

Monitoring of Genetic Abnormalities

The polynucleotides of the preceding paragraphs have a number of different specific uses. The human genes set forth in FIG. 1 or FIG. 2 map to the chromosomal locations set forth in Example 2. For example, because a FIG. 1 or FIG. 2 gene maps to a particular chromosome, polynucleotides that encode different regions of the gene of FIG. 1 or FIG. 2-related proteins are used to characterize cytogenetic abnormalities of this chromosomal locale, such as abnormalities that are identified as being associated with various cancers. In certain genes, a variety of chromosomal abnormalities including rearrangements have been identified as frequent cytogenetic abnormalities in a number of different cancers (see e.g. Krajinovic et al., Mutat. Res. 382(3-4): 81-83 (1998); Johansson et al., Blood 86(10): 3905-3914 (1995) and Finger et al., P.N.A.S. 85(23): 9158-9162 (1988)). Thus, polynucleotides encoding specific regions of the gene of FIG. 1 or FIG. 2-related proteins provide new tools that can be used to delineate, with greater precision than previously possible, cytogenetic abnormalities in the chromosomal regions discussed in Example 2, and how they contribute to the malignant phenotype. In this context, these polynucleotides satisfy a need in the art for expanding the sensitivity of chromosomal screening in order to identify more subtle and less common chromosomal abnormalities (see e.g. Evans et al., Am. J. Obstet. Gynecol 171(4): 1055-1057 (1994)).

Furthermore, as the genes set forth in FIG. 1 or FIG. 2 are shown to be highly expressed in cancers, the FIG. 1 or FIG. 2 polynucleotides are used in methods assessing the status of the nucleic acid sequence of FIG. 1 or FIG. 2 expression in normal versus cancerous tissues. Typically, polynucleotides that encode specific regions of the gene of FIG. 1 or FIG. 2-related proteins are used to assess the presence of perturbations (such as deletions, insertions, point mutations, or alterations resulting in a loss of an antigen etc.) in specific regions of the FIG. 1 or FIG. 2 genes, such as regions containing one or more motifs. Exemplary assays include both RT-PCR assays as well as single-strand conformation polymorphism (SSCP) analysis (see, e.g., Marrogi et al., J. Cutan. Pathol. 26(8): 369-378 (1999), both of which utilize polynucleotides encoding specific regions of a protein to examine these regions within the protein.

Antisense Embodiments

Other specifically contemplated nucleic acid related embodiments of the invention disclosed herein are genomic DNA, cDNAs, ribozymes, and antisense molecules, as well as nucleic acid molecules based on an alternative backbone, or including alternative bases, whether derived from natural sources or synthesized, and include molecules capable of inhibiting the RNA or protein expression of a gene set forth in FIG. 1 or FIG. 2. For example, antisense molecules can be RNAs or other molecules, including peptide nucleic acids (PNAs) or non-nucleic acid molecules such as phosphorothioate derivatives that specifically bind DNA or RNA in a base pair-dependent manner. A skilled artisan can readily obtain these classes of nucleic acid molecules using the FIG. 1 or FIG. 2 polynucleotides and polynucleotide sequences disclosed herein.

Antisense technology entails the administration of exogenous oligonucleotides that bind to a target polynucleotide located within the cells. The term “antisense” refers to the fact that such oligonucleotides are complementary to their intracellular targets, (e.g., a gene of FIG. 1 or FIG. 2). See for example, Jack Cohen, Oligodeoxynucleotides, Antisense Inhibitors of Gene Expression, CRC Press, 1989; and Synthesis 1:1-5 (1988). The FIG. 1 or FIG. 2 antisense oligonucleotides of the present invention include derivatives such as S-oligonucleotides (phosphorothioate derivatives or S-oligos, see, Jack Cohen, supra), which exhibit enhanced cancer cell growth inhibitory action. S-oligos (nucleoside phosphorothioates) are isoelectronic analogs of an oligonucleotide (O-oligo) in which a nonbridging oxygen atom of the phosphate group is replaced by a sulfur atom. The S-oligos of the present invention can be prepared by treatment of the corresponding O-oligos with 3H-1,2-benzodithiol-3-one-1,1-dioxide, which is a sulfur transfer reagent. See, e.g., Iyer, R. P. et al., J. Org. Chem. 55:4693-4698 (1990); and Iyer, R. P. et al, J. Am. Chem. Soc. 112:1253-1254 (1990). Additionally, the FIG. 1 or FIG. 2 antisense oligonucleotides of the present invention include morpholino antisense oligonucleotides known in the art (see, e.g., Partridge et al., 1996, Antisense & Nucleic Acid Drug Development 6: 169-175).

The FIG. 1 or FIG. 2 antisense oligonucleotides of the present invention typically can be RNA or DNA that is complementary to and stably hybridizes with the first 100.5′ codons or last 100 3′ codons of a genomic sequence or the corresponding mRNA of the invention. Absolute complementarity is not required, although high degrees of complementarity are preferred. Use of an oligonucleotide complementary to this region allows for the selective hybridization to mRNA of the invention and not to mRNA specifying other regulatory subunits of protein kinase. In one embodiment, the FIG. 1 or FIG. 2 antisense oligonucleotides of the present invention are 15 to 30-mer fragments of the antisense DNA molecule that have a sequence that hybridizes to mRNA of the invention. Optionally, a FIG. 1 or FIG. 2 antisense oligonucleotide is a 30-mer oligonucleotide that is complementary to a region in the first 10 5′ codons or last 10 3′ codons of a gene set forth in FIG. 1 or FIG. 2. Alternatively, the antisense molecules are modified to employ ribozymes in the inhibition of expression of a gene set forth in FIG. 1 or FIG. 2, see, e.g., L. A. Couture & D. T. Stinchcomb; Trends Genet 12: 510-515 (1996).

Primers and Primer Pairs

Further specific embodiments of the nucleotides of the invention include primers and primer pairs, which allow the specific amplification of polynucleotides of the invention or of any specific parts thereof, and probes that selectively or specifically hybridize to nucleic acid molecules of the invention or to any part thereof Probes can be labeled with a detectable marker, such as, for example, a radioisotope, fluorescent compound, bioluminescent compound, a chemiluminescent compound, metal chelator or enzyme. Such probes and primers are used to detect the presence of a FIG. 1 or FIG. 2 polynucleotide in a sample and as a means for detecting a cell expressing a gene of FIG. 1 or FIG. 2-related protein.

Examples of such probes include polynucleotides comprising all or part of a human gene set forth in FIG. 1 or FIG. 2. Examples of primer pairs capable of specifically amplifying an mRNA of the invention are also disclosed herein. As will be understood by the skilled artisan, a great many different primers and probes can be prepared based on the sequences provided herein and used effectively to amplify and/or detect an mRNA of the invention.

The FIG. 1 or FIG. 2 polynucleotides of the invention are useful for a variety of purposes, including but not limited to their use as probes and primers for the amplification and/or detection of the FIG. 1 or FIG. 2 gene(s), mRNA(s), or fragments thereof; as reagents for the diagnosis and/or prognosis of prostate cancer and other cancers; as coding sequences capable of directing the expression of a gene of FIG. 1 or FIG. 2-related polypeptide; as tools for modulating or inhibiting the expression of a FIG. 1 or FIG. 2 gene(s) and/or translation of a FIG. 1 or FIG. 2 transcript(s); and as therapeutic agents.

The present invention includes the use of any probe as described herein to identify and isolate a gene set forth in FIG. 1 or FIG. 2, or FIG. 1 or FIG. 2-related nucleic acid sequence of the invention from a naturally occurring source, such as humans or other mammals, as well as the isolated nucleic acid sequence per se, which would comprise all or most of the sequences found in the probe used.

Isolation of Nucleic Acid Molecules

The cDNA sequences described herein (e.g., FIG. 1 or FIG. 2), enable the isolation of other polynucleotides of the invention, as well as the isolation of polynucleotides encoding homologs of a protein corresponding to a gene of FIG. 1 or FIG. 2, alternatively spliced isoforms, allelic variants, and mutant forms of a gene product of a gene of the invention as well as polynucleotides that encode analogs of the gene of FIG. 1 or FIG. 2-related proteins. Various molecular cloning methods that can be employed to isolate full length cDNAs encoding a FIG. 1 or FIG. 2 gene are well known (see, for example, Sambrook, J. et al., Molecular Cloning: A Laboratory Manual, 2d edition, Cold Spring Harbor Press, New York, 1989; Current Protocols in Molecular Biology. Ausubel et al., Eds., Wiley and Sons, 1995). For example, lambda phage cloning methodologies can be conveniently employed, using commercially available cloning systems (e.g., Lambda ZAP Express, Stratagene). Phage clones containing a FIG. 1 or FIG. 2 gene cDNA can be identified by probing with a labeled cDNA of FIG. 1 or FIG. 2 or a fragment thereof. For example, in one embodiment, a FIG. 1 or FIG. 2 cDNA or a portion thereof is synthesized and used as a probe to retrieve overlapping and full-length cDNAs corresponding to a gene set forth in FIG. 1 or FIG. 2. A gene set forth in FIG. 1 or FIG. 2 itself can be isolated by screening genomic DNA libraries, bacterial artificial chromosome libraries (BACs), yeast artificial chromosome libraries (YACs), and the like, with a respective gene in FIG. 1 or FIG. 2 DNA probe or primer.

Recombinant Nucleic Acid Molecules and Host-Vector Systems

The invention also provides recombinant DNA or RNA molecules containing a polynucleotide, a fragment, analog or homologue thereof in accordance with the invention, including but not limited to phages, plasmids, phagemids, cosmids, YACs, BACs, as well as various viral and non-viral vectors well known in the art, and cells transformed or transfected with such recombinant DNA or RNA molecules. Methods for generating such molecules are well known (see, for example, Sambrook et al, 1989, supra).

The invention further provides a host-vector system comprising a recombinant DNA molecule containing polynucleotide (fragment, analog or homologue thereof) in accordance with the invention within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as a Sf9 or HighFive cell). Examples of suitable mammalian cells include various prostate cancer cell lines such as DU145 and TsuPr1, other transfectable or transducible prostate cancer cell lines, primary cells (PrEC), as well as a number of mammalian cells routinely used for the expression of recombinant proteins (e.g., COS, CHO, 293, 293T cells).

A wide range of host-vector systems suitable for the expression of gene of FIG. 1 or FIG. 2-related proteins or fragments thereof are available, see for example, Sambrook et al., 1989, supra; Current Protocols in Molecular Biology, 1995, supra). Preferred vectors for mammalian expression include but are not limited to pcDNA 3.1 myc-His-tag (Invitrogen) and the retroviral vector pSRαtkneo (Muller et al., 1991, MCB 11:1785). Using these expression vectors, proteins of the invention can be expressed in several prostate cancer and non-prostate cell lines, including for example 293, 293T, rat-1, NIH 3T3 and TsuPr1. The host-vector systems of the invention are useful for the production of a gene of FIG. 1 or FIG. 2-related protein or fragment thereof. Such host-vector systems can be employed to study the functional properties of a protein of the invention and related mutations or analogs.

Recombinant human gene of FIG. 1 or FIG. 2-related proteins, or an analog or homolog or fragment thereof can be produced by mammalian cells transfected with a construct containing a FIG. 1 or FIG. 2-related nucleotide. For example, 293T cells can be transfected with an expression plasmid encoding a gene of FIG. 1 or FIG. 2-related protein or fragment, analog or homolog thereof, a gene of FIG. 1 or FIG. 2-related protein is expressed in the 293T cells, and the recombinant gene of FIG. 1 or FIG. 2-related protein is isolated using standard purification methods (e.g., affinity purification using antibodies of the invention, e.g., an antibody that specifically binds a gene of FIG. 1 or FIG. 2-related protein, i.e., a protein corresponding to a gene set forth in FIG. 1 or FIG. 2). In another embodiment, a FIG. 1 or FIG. 2 coding sequence is subcloned into the retroviral vector pSRαMSVtkneo and used to infect various manmalian cell lines, such as NIH 3T3, TsuPr1, 293 and rat-1 in order to establish cell lines that express a gene of the invention. Various other expression systems well known in the art can also be employed. Expression constructs encoding a leader peptide joined in frame to a FIG. 1 or FIG. 2 coding sequence can be used for the generation of a secreted form of recombinant gene of FIG. 1 or FIG. 2-related proteins.

As discussed herein, redundancy in the genetic code permits variation in the gene sequences set forth in FIG. 1 or FIG. 2. In particular, it is known in the art that specific host species often have specific codon preferences, and thus one can adapt the disclosed sequence as preferred for a desired host. For example, preferred analog codon sequences typically have rare codons (i.e., codons having a usage frequency of less than about 20% in known sequences of the desired host) replaced with higher frequency codons. Codon preferences for a specific species are calculated, for example, by utilizing codon usage tables available on the INTERNET such as at URL www.dna.affrc.go jp/˜nakamura/codon.html.

Additional sequence modifications are known to enhance protein expression in a cellular host. These include elimination of sequences encoding spurious polyadenylation signals, exon/intron splice site signals, transposon-like repeats, and/or other such well-characterized sequences that are deleterious to gene expression. The GC content of the sequence is adjusted to levels average for a given cellular host, as calculated by reference to known genes expressed in the host cell. Where possible, the sequence is modified to avoid predicted hairpin secondary mRNA structures. Other useful modifications include the addition of a translational initiation consensus sequence at the start of the open reading frame, as described in Kozak, Mol. Cell Biol., 9:5073-5080 (1989). Skilled artisans understand that the general rule that eukaryotic ribosomes initiate translation exclusively at the 5′ proximal AUG codon is abrogated only under rare conditions (see, e.g., Kozak PNAS 92(7): 2662-2666, (1995) and Kozak NAR 15(20): 8125-8148 (1987)).

Gene of FIG. 1 or FIG. 2-related Proteins

Another aspect of the present invention provides gene of FIG. 1 or FIG. 2-related proteins, i.e., proteins of the invention. Alternatively, embodiments of a gene of FIG. 1 or FIG. 2-related protein comprise variant, homolog or analog polypeptides that have alterations in their amino acid sequence relative to a native protein.

Embodiments of the invention disclosed herein include a wide variety of art-accepted variants or analogs of a gene of FIG. 1 or FIG. 2-related proteins such as polypeptides having amino acid insertions, deletions and substitutions. FIG. 1 or FIG. 2 variants can be made using methods known in the art such as site-directed mutagenesis, alanine scanning, and PCR mutagenesis. Site-directed mutagenesis (Carter et al., Nucl. Acids Res., 13:4331 (1986); Zoller et al., NucL Acids Res., 10:6487 (1987)), cassette mutagenesis (Wells et al, Gene, 34:315 (1985)), restriction selection mutagenesis (Wells et al., Philos. Trans. R. Soc. London SerA, 317:415 (1986)) or other known techniques can be performed on the cloned DNA to produce variant DNA in accordance with the invention.

As defined herein, gene of FIG. 1 or FIG. 2-related protein variants, analogs or homologs, have the distinguishing attribute of having at least one epitope that is “cross reactive” with a gene of FIG. 1 or FIG. 2-related protein. As used in this sentence, “cross reactive” means that an antibody or T cell that specifically binds to a FIG. 1 or FIG. 2-related protein variant also specifically binds to a gene of FIG. 1 or FIG. 2-related protein. A polypeptide ceases to be a variant of its “parental” polypeptide, when it no longer contains any epitope capable of being recognized by an antibody or T cell that specifically binds to the parental polypeptide. Those skilled in the art understand that antibodies that recognize proteins bind to epitopes of varying size, and a grouping of the order of about four or five amino acids, contiguous or not, is regarded as a typical number of amino acids in a minimal epitope. See, e.g., Nair et al., J. Immunol 2000 165(12): 6949-6955; Hebbes et al., Mol Immunol (1989) 26(9):865-73; Schwartz et al., J Immunol (1985) 135(4):2598-608.

Other classes of a gene of FIG. 1 or FIG. 2-related protein variants share 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more similarity, homology or identity with a nucleic acid sequence of FIG. 1 or FIG. 2, or a fragment thereof. Another specific class of a gene of FIG. 1 or FIG. 2-related protein variants or analogs comprise one or more of the biological motifs described herein or presently known in the art. It is to be appreciated that motifs now or which become part of the art are to be applied to the nucleic, or corresponding amino acid sequences, of FIG. 1 or FIG. 2.

As discussed herein, embodiments of the claimed invention include polypeptides containing less than the full nucleic acid sequence of a gene shown in FIG. 1 or FIG. 2. For example, representative embodiments of the invention comprise nucleic acid sequences having any: 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1025, 1050, 1075, 1100, 1125, 1150, 1175, 1200, etc., or more contiguous nucleic acids of a sequence shown in FIG. 1 or FIG. 2.

Motif-Bearing Protein Embodiments

Additional illustrative embodiments of the invention disclosed herein include polypeptides of the invention that comprise the amino acid residues of one or more of the biological motifs contained within a gene of FIG. 1 or FIG. 2-related protein polypeptide sequence of the invention. Various motifs are known in the art, and a protein can be evaluated for the presence of such motifs by a number of publicly available Internet sites (see, e.g., World Wide Web URL addresses: pfam.wust1.edu/; searchlauncher.bcm.tmc.edu/seq-searcb /struc-predict.html; psort.ims.u-tokyo.acjp/; www.cbs.dtu.dk/; www.ebi.ac.uk/interpro/scan.html; www.expasy.ch/tools/scnpsitl.htrnl; Epimatrix™ and Epimer™, Brown University, www.brown.edu/Research/TB-HIV_Lab/epimatrix/epimatrix.html; and BIMAS, bimas.dcrt.nih.gov/.).

Polypeptides comprising one or more of the motifs in the art are useful in elucidating the specific characteristics of a malignant phenotype in view of the observation that the motifs discussed above are associated with growth dysregulation and because the proteins of the invention are overexpressed in certain cancers (See, e.g., Table I). Casein kinase II, cAMP and camp-dependent protein kinase, and Protein Kinase C, for example, are enzymes known to be associated with the development of the malignant phenotype (see e.g. Chen et al, Lab Invest., 78(2): 165-174 (1998); Gaiddon et al., Endocrinology 136(10): 4331-4338 (1995); Hall et al, Nucleic Acids Research 24(6): 1119-1126 (1996); Peterziel et al., Oncogene 18(46): 6322-6329 (1999) and O'Brian, Oncol. Rep. 5(2): 305-309 (1998)). Moreover, both glycosylation and myristoylation are protein modifications also associated with cancer and cancer progression (see e.g. Dennis et al., Biochem. Biophys. Acta 1473(l):21-34 (1999); Raju et al., Exp. Cell Res. 235(1): 145-154 (1997)). Amidation is another protein modification also associated with cancer and cancer progression (see e.g. Treston et al., J. Natl. Cancer Inst. Monogr. (13): 169-175 (1992)).

Gene of FIG. 1 or FIG. 2-related proteins are embodied in many forms, preferably in isolated form. A purified gene of FIG. 1 or FIG. 2-related protein molecule will be substantially free of other proteins or molecules that impair the binding of a gene of FIG. 1 or FIG. 2-related protein to an antibody, T cell or other ligand. The nature and degree of isolation and purification will depend on the intended use. Embodiments of a gene of FIG. 1 or FIG. 2-related proteins include purified gene of FIG. 1 or FIG. 2-related proteins and functional, soluble gene of FIG. 1 or FIG. 2-related proteins. In one embodiment, a functional, soluble gene of FIG. 1 or FIG. 2-related protein or fragment thereof retains the ability to be bound by an antibody, T cell or other ligand.

FIG. 1 or FIG. 2-related polypeptides that contain particularly interesting structures can be predicted and/or identified using various analytical techniques well known in the art, including, for example, the methods of Chou-Fasman, Garnier-Robson, Kyte-Doolittle, Eisenberg, Karplus-Schultz or Jameson-Wolf analysis, or on the basis of immunogenicity. Fragments that contain such structures are particularly useful in generating subunit-specific antibodies that bind to a gene of FIG. 1 or FIG. 2-related protein, or T cells or in identifying cellular factors that bind to a protein of the invention. For example, hydrophilicity profiles can be generated, and immunogenic peptide fragments identified, using the method of Hopp, T. P. and Woods, K. R. , 1981, Proc. Natl. Acad. Sci. U.S.A. 78:3824-3828. Hydropathicity profiles can be generated, and immunogenic peptide fragments identified, using the method of Kyte, J. and Doolittle, R. F. , 1982, J. Mol. Biol. 157:105-132. Percent (%) Accessible Residues profiles can be generated, and immunogenic peptide fragments identified, using the method of Janin J. , 1979, Nature 277:491-492. Average Flexibility profiles can be generated, and immunogenic peptide fragments identified, using the method of Bhaskaran R., Ponnuswamy P. K. , 1988, Int. J. Pept. Protein Res. 32:242-255. Beta-turn profiles can be generated, and immunogenic peptide fragments identified, using the method of Deleage, G., Roux B., 1987, Protein Engineering 1:289-294.

The HLA peptide motif search algorithm was developed by Dr. Ken Parker based on binding of specific peptide sequences in the groove of HLA Class I molecules, in particular HLA-A2 (see, e.g., Falk et al Nature 351: 290-6 (1991); Hunt et al., Science 255:1261-3 (1992); Parker et al J. Immunol. 149:3580-7 (1992); Parker et al., J. Immunol. 152:163-75 (1994)). This algorithm allows location and ranking of 8-mer, 9-mer, and 10-mer peptides from a complete protein sequence for predicted binding to HLA-A2 as well as numerous other HLA Class I molecules. Many HLA class I binding peptides are 8-, 9-, 10 or 11-mers. For example, for class I HLA-A2, the epitopes preferably contain a leucine (L) or methionine (M) at position 2 and a valine (V) or leucine (L) at the C-terminus (see, e.g., Parker et al., J. Immunol. 149:3580-7 (1992)).

Actual binding of peptides to an HLA allele can be evaluated by stabilization of HLA expression on the antigen-processing defective cell line T2 (see, e.g., Xue et al., Prostate 30:73-8 (1997) and Peshwa et al, Prostate 36:129-38 (1998)). Immunogenicity of specific peptides can be evaluated in vitro by stimulation of CD8+cytotoxic T lymphocytes (CTL) in the presence of antigen presenting cells such as dendritic cells.

It is to be appreciated that every epitope predicted by the BIMAS site, Epimer™ and Epimatrix™ sites, or specified by the HLA class I or class II motifs available in the art or which become part of the art (or determined using World Wide Web site URL syfpeithi.bmi-heidelberg.com/, or BIMAS, bimas.dcrt.nih.gov/) are to be “applied” to a gene of FIG. 1 or FIG. 2-related protein in accordance with the invention. As used in this context “applied” means that a gene of FIG. 1 or FIG. 2-related protein is evaluated, e.g., visually or by computer-based patterns finding methods, as appreciated by those of skill in the relevant art. Every subsequence of a gene of FIG. 1 or FIG. 2-related protein of 8, 9, 10, or 11 amino acid residues that bears an HLA Class I motif, or a subsequence of 9 or more amino acid residues that bear an HLA Class II motif are within the scope of the invention.

Expression of Gene of FIG. 1 or FIG. 2-Related Proteins

In an embodiment described in the examples that follow, the proteins of the invention can be conveniently expressed in cells (such as 293T cells) transfected with a commercially available expression vector such as a CMV-driven expression vector encoding a gene of FIG. 1 or FIG. 2-related protein with a C-terminal 6XHis and MYC tag (pcDNA3.1/mycHIS, Invitrogen or Tag5, GenHunter Corporation, Nashville Tenn.). The Tag5 vector provides an IgGK secretion signal that can be used to facilitate the production of a secreted gene of FIG. 1 or FIG. 2-related protein in transfected cells. A secreted HIS-tagged gene of FIG. 1 or FIG. 2-related protein in the culture media can be purified, e.g., using a nickel column using standard techniques.

Modifications of Gene of FIG. 1 or FIG. 2-Related Proteins

Modifications of gene of FIG. 1 or FIG. 2-related proteins such as covalent modifications are included within the scope of this invention. One type of covalent modification includes reacting targeted amino acid residues of a gene of FIG. 1 or FIG. 2-related protein polypeptide with an organic derivatizing agent that is capable of reacting with selected side chains or the N- or C- terminal residues of a gene of FIG. 1 or FIG. 2-related protein. Another type of covalent modification to a gene of FIG. 1 or FIG. 2-related protein polypeptide included within the scope of this invention comprises altering the native glycosylation pattern of a protein related to a gene of FIG. 1 or FIG. 2. Another type of covalent modification to a gene of FIG. 1 or FIG. 2-related protein comprises linking a polypeptide of the invention to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

Gene of FIG. 1 or FIG. 2-related proteins of the present invention can also be modified to form a chimeric molecule comprising a gene of FIG. 1 or FIG. 2-related protein fused to another, heterologous polypeptide or amino acid sequence. Such a chimeric molecule can be synthesized chemically or recombinantly. A chimeric molecule can have a gene of FIG. 1 or FIG. 2-related protein fused to another tumor-associated antigen or fragment thereof. Alternatively, a protein in accordance with the invention can comprise a fusion of fragments of a FIG. 1 or FIG. 2 sequence (amino or nucleic acid) such that a molecule is created that is not, through its length, directly homologous to the nucleic acid sequences shown in FIG. 1 or FIG. 2. A chimeric molecule can comprise a fusion of a FIG. 1 or FIG. 2-related protein with a polyhistidine epitope tag, which provides an epitope to which immobilized nickel can selectively bind, with cytokines or with growth factors. The epitope tag is generally placed at the amino- or carboxyl- terminus of a gene of FIG. 1 or FIG. 2-related protein. In an alternative embodiment, the chimeric molecule can comprise a fusion of a FIG. 1 or FIG. 2-related protein with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a FIG. 1 or FIG. 2 polypeptide in place of at least one variable region within an Ig molecule. In a preferred embodiment, the immunoglobulin fusion includes the hinge, CH2 and CH3, or the hinge, CHI, CH2 and CH3 regions of an IgGI molecule. For the production of immunoglobulin fusions see, e.g., U.S. Patent No. 5,428,130 issued Jun. 27, 1995.

Uses of Gene of FIG. 1 or FIG. 2-Related Proteins

Gene of FIG. 1 or FIG. 2-related protein fragments/subsequences are particularly useful in generating and characterizing domain-specific antibodies (e.g., antibodies recognizing an extracellular or intracellular epitope of a gene of FIG. 1 or FIG. 2-related protein), for identifying agents or cellular factors that bind to a protein of the invention or a particular structural domain thereof, and in various therapeutic and diagnostic contexts, including but not limited to diagnostic assays, cancer vaccines and methods of preparing such vaccines.

Proteins encoded by a gene of the invention (e.g., a FIG. 1 or FIG. 2 gene, or analog, homolog or fragment thereof) have a variety of uses, including but not limited to generating antibodies and in methods. for identifying ligands and other agents and cellular constituents that bind to a FIG. 1 or FIG. 2 gene product. Antibodies raised against a gene of FIG. 1 or FIG. 2-related protein or fragment thereof are useful in diagnostic and prognostic assays, and imaging methodologies in the management of human cancers characterized by expression of a gene of FIG. 1 or FIG. 2-related protein, such as those listed in Table I. Such antibodies can be expressed intracellularly and used in methods of treating patients with such cancers. FIG. 1 or FIG. 2-related nucleic acids or proteins are also used in generating HTL or CTL responses.

Various immunological assays useful for the detection of gene of FIG. 1 or FIG. 2-related proteins are used, including but not limited to various types of radioimmunoassays, enzyme-linked immunosorbent assays (ELISA), enzyme-linked immunofluorescent assays (ELIFA), immunocytochemical methods, and the like. Antibodies can be labeled and used as immunological imaging reagents capable of detecting cells that express a gene of the invention (e.g., in radioscintigraphic imaging methods). Gene of FIG. 1 or FIG. 2-related proteins are also particularly useful in generating cancer vaccines, as further described herein.

Transgenic Animals of the Invention

Nucleic acids that encode a gene of FIG. 1 or FIG. 2-related protein can also be used to generate either transgenic animals or “knock out” animals that, in turn, are useful in the development and screening of therapeutically useful reagents. In accordance with established techniques, cDNA encoding a gene of FIG. 1 or FIG. 2-related protein can be used to clone genomic DNA that encodes a gene of FIG. 1 or FIG. 2-related protein. The cloned genomic sequences can then be used to generate transgenic animals containing cells that express DNA that encode a gene of FIG. 1 or FIG. 2-related protein. Methods for generating transgenic animals, particularly animals such as mice or rats, have become conventional in the art and are described, for example, in U.S. Pat. Nos. 4,736,866 issued Apr. 12, 1988, and 4,870,009 issued Sep. 26, 1989. Typically, particular cells would be targeted for a nucleic acid sequence of a gene of FIG. 1 or FIG. 2 transgene incorporation with tissue-specific enhancers.

Transgenic animals that include a copy of a transgene encoding a gene of FIG. 1 or FIG. 2-related protein can be used to examine the effect of increased expression of DNA that encodes the gene of FIG. 1 or FIG. 2-related protein. Such animals can be used as tester animals for reagents thought to confer protection from, for example, pathological conditions associated with its overexpression. In accordance with this aspect of the invention, an animal is treated with a reagent and a reduced incidence of a pathological condition, compared to untreated animals that bear the transgene, would indicate a potential therapeutic intervention for the pathological condition.

Alternatively, non-human homologues of gene of FIG. 1 or FIG. 2-related proteins can be used to construct a gene of FIG. 1 or FIG. 2-related protein “knock out” animal that has a defective or altered gene encoding the gene of FIG. 1 or FIG. 2-related protein as a result of homologous recombination between the endogenous gene encoding the gene of FIG. 1 or FIG. 2-related protein and altered genomic DNA encoding the gene of FIG. 1 or FIG. 2-related protein, introduced into an embryonic cell of the animal. For example, cDNA that encodes a gene of FIG. 1 or FIG. 2-related protein can be used to clone genomic DNA encoding the gene of FIG. 1 or FIG. 2-related protein, in accordance with established techniques. A portion of the genomic DNA encoding a gene of FIG. 1 or FIG. 2-related protein can be deleted or replaced with another gene, such as a gene encoding a selectable marker that can be used to monitor integration. Typically, several kilobases of unaltered flanking DNA (both at the 5′ and 3′ ends) are included in the vector (see, e.g., Thomas and Capecchi, Cell, 51:503 (1987) for a description of homologous recombination vectors). The vector is introduced into an embryonic stem cell line (e.g., by electroporation) and cells in which the introduced DNA has homologously recombined with the endogenous DNA are selected (see, e.g., Li et al., Cell, 69:915 (1992)). The selected cells are then injected into a blastocyst of an animal (e.g., a mouse or rat) to form aggregation chimeras (see, e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal, and the embryo brought to term to create a “knock out” animal. Progeny harboring the homologously recombined DNA in their germ cells can be identified by standard techniques and used to breed animals in which all cells of the animal contain the homologously recombined DNA. Knock out animals can be characterized, for example, for their ability to defend against certain pathological conditions or for their development of pathological conditions due to absence of a gene of FIG. 1 or FIG. 2-related protein.

Methods for the Detection of a Gene or Gene Products of the Invention

Another aspect of the present invention relates to methods for detecting FIG. 1 or FIG. 2 polynucleotides and FIG. 1 or FIG. 2-related gene products, as well as methods for identifying a cell that expresses a gene set forth in FIG. 1 or FIG. 2. The expression profile of a gene in FIG. 1 or FIG. 2 makes it a diagnostic marker for metastasized disease. Accordingly, the status of FIG. 1 or FIG. 2 gene products provides information useful for predicting a variety of factors including susceptibility to advanced stage disease, rate of progression, and/or tumor aggressiveness. As discussed in detail herein, the status of FIG. 1 or FIG. 2 gene products in patient samples can be analyzed by a variety protocols that are well known in the art including immunohistochemical analysis, the variety of Northern blotting techniques including in situ hybridization, RT-PCR analysis (for example on laser capture micro-dissected samples), Western blot analysis and tissue array analysis.

More particularly, the invention provides assays for the detection of FIG. 1 or FIG. 2 polynucleotides in a biological sample, such as serum, bone, prostate, and other tissues, urine, semen, cell preparations, and the like. Detectable FIG. 1 or FIG. 2 polynucleotides include, for example, a FIG. 1 or FIG. 2 gene or fragment thereof, a FIG. 1 or FIG. 2 mRNA, alternative splice variants of FIG. 1 or FIG. 2 mRNAs, and recombinant DNA or RNA molecules that contain a FIG. 1 or FIG. 2 polynucleotide. A number of methods for amplifying and/or detecting the presence of FIG. 1 or FIG. 2 polynucleotides are well known in the art and can be employed in the practice of this aspect of the invention.

In one embodiment, a method for detecting an a FIG. 1 or FIG. 2 mRNA in a biological sample comprises producing cDNA from the sample by reverse transcription using at least one primer; amplifying the cDNA so produced using FIG. 1 or FIG. 2 polynucleotides as sense and antisense primers to amplify FIG. 1 or FIG. 2 cDNAs therein; and detecting the presence of the amplified FIG. 1 or FIG. 2 cDNA. Optionally, the sequence of the amplified FIG. 1 or FIG. 2 cDNA can be determined.

In another embodiment, a method of detecting a FIG. 1 or FIG. 2 gene in a biological sample comprises first isolating genomic DNA from the sample; amplifying the isolated genomic DNA using FIG. 1 or FIG. 2 polynucleotides as sense and antisense primers; and detecting the presence of the amplified FIG. 1 or FIG. 2 gene. Any number of appropriate sense and antisense probe combinations can be designed from a FIG. 1 or FIG. 2 nucleotide sequence and used for this purpose.

The invention also provides assays for detecting the presence of a gene of FIG. 1 or FIG. 2-related protein in a tissue or other biological sample such as serum, semen, bone, prostate, urine, cell preparations, and the like. Methods for detecting a gene of FIG. 1 or FIG. 2-related protein are also well known and include, for example, immunoprecipitation, immunohistochemical analysis, Western blot analysis, molecular binding assays, ELISA, ELIFA and the like. For example, a method of detecting the presence of a gene of FIG. 1 or FIG. 2-related protein in a biological sample comprises first contacting the sample with a FIG. 1 or FIG. 2-related antibody, a FIG. 1 or FIG. 2-reactive fragment thereof, or a recombinant protein containing an antigen binding region of a FIG. 1 or FIG. 2-related antibody; and then detecting the binding of a FIG. 1 or FIG. 2-related protein in the sample.

Methods for identifying a cell that expresses a gene of FIG. 1 or FIG. 2 are also within the scope of the invention. In one embodiment, an assay for identifying a cell that expresses a FIG. 1 or FIG. 2 gene comprises detecting the presence of a FIG. 1 or FIG. 2 mRNA in the cell. Methods for the detection of particular mRNAs in cells are well known and include, for example, hybridization assays using complementary DNA probes (such as in situ hybridization using labeled riboprobes to a gene of FIG. 1 or FIG. 2, Northern blot and related techniques) and various nucleic acid amplification assays (such as RT-PCR using complementary primers specific for genes of FIG. 1 or FIG. 2, and other amplification type detection methods, such as, for example, branched DNA, SISBA, TMA and the like). Alternatively, an assay for identifying a cell that expresses a FIG. 1 or FIG. 2 gene comprises detecting the presence of a FIG. 1 or FIG. 2-related protein in the cell or secreted by the cell. Various methods for the detection of proteins are well known in the art and are employed for the detection of gene of FIG. 1 or FIG. 2-related proteins and cells that express FIG. 1 or FIG. 2-related proteins. Expression analysis of gene of FIG. 1 or FIG. 2-related proteins is also useful as a tool for identifying and evaluating agents that modulate FIG. 1 or FIG. 2 gene expression. For example, FIG. 1 or FIG. 2 gene expression is significantly upregulated in prostate cancer, and is expressed in cancers of the tissues listed in Table I. Identification of a molecule or biological agent that inhibits FIG. 1 or FIG. 2 gene expression or over-expression in cancer cells is of therapeutic value. For example, such an agent can be identified by using a screen that quantifies a FIG. 1 or FIG. 2 gene expression by RT-PCR, nucleic acid hybridization or antibody binding.

Methods for Monitoring the Status of Genes of the Invention

Oncogenesis is known to be a multistep process where cellular growth becomes progressively dysregulated and cells progress from a normal physiological state to precancerous and then cancerous states (see, e.g., Alers et al, Lab Invest. 77(5): 437-438 (1997) and Isaacs et al, Cancer Surv. 23: 19-32 (1995)). In this context, examining a biological sample for evidence of dysregulated cell growth (such as aberrant gene of FIG. 1 or FIG. 2 expression in cancers) allows for early detection of such aberrant physiology, before a pathologic state such as cancer has progressed to a stage that therapeutic options are more limited and or the prognosis is worse. In such examinations, the status of the genes in FIG. 1 or FIG. 2 in a biological sample of interest can be compared, for example, to the status of that gene of FIG. 1 or FIG. 2 in a corresponding normal sample (e.g. a sample from that individual or alternatively another individual that is not affected by a pathology). An alteration in the status of a gene of FIG. 1 or FIG. 2 in the biological sample (as compared to the normal sample) provides evidence of dysregulated cellular growth. In addition to using a biological sample that is not affected by a pathology as a normal sample, one can also use a predetermined normative value such as a predetermined normal level of mRNA expression (see, e.g., Grever et al., J. Comp. Neurol. Dec. 9, 1996 ; 376(2): 306-14 and U.S. Pat. No. 5,837,501) to compare the status of a gene or protein in a sample.

The term “status” in this context is used according to its art accepted meaning and refers to the condition or state of a gene and its products. Typically, skilled artisans use a number of parameters to evaluate the condition or state of a gene and its products. These include, but are not limited to the location of expressed gene products (including the location of gene of FIG. 1 or FIG. 2 expressing cells) as well as the level, and biological activity of expressed gene products (such as FIG. 1 or FIG. 2 mRNA, polynucleotides and polypeptides). Typically, an alteration in the status of a gene of FIG. 1 or FIG. 2, and/or a gene of FIG. 1 or FIG. 2-related protein, comprises a change in the location of a FIG. 1 or FIG. 2-related protein and/or of cells that express a gene of FIG. 1 or FIG. 2 and/or an increase in FIG. 1 or FIG. 2 mRNA and/or protein expression.

The status in a sample of a gene of FIG. 1 or FIG. 2 or a gene of FIG. 1 or FIG. 2-related protein can be analyzed by a number of means well known in the art, including without limitation, immunohistochemical analysis, in situ hybridization, RT-PCR analysis on laser capture micro-dissected samples, Western blot analysis, and tissue array analysis. Typical protocols for evaluating the status of a FIG. 1 or FIG. 2 gene and gene products are found, for example in Ausubel et al eds., 1995, Current Protocols In Molecular Biology, Units 2 (Northern Blotting), 4 (Southern Blotting), 15 (Immunoblotting) and 18 (PCR Analysis). Thus, the status of a gene or protein of FIG. 1 or FIG. 2 in a biological sample is evaluated by various methods utilized by skilled artisans including, but not limited to genomic Southern analysis (to examine, for example perturbations in a FIG. 1 or FIG. 2 gene), Northern analysis and/or PCR analysis of FIG. 1 or FIG. 2 mRNA (to examine, for example alterations in the polynucleotide sequences or expression levels of FIG. 1 or FIG. 2 mRNAs), and, Western and/or immunohistochemical analysis (to examine, for example alterations in polypeptide sequences, alterations in polypeptide localization within a sample, alterations in expression levels of gene of FIG. 1 or FIG. 2-related proteins and/or associations of gene of FIG. 1 or FIG. 2-related proteins with polypeptide binding partners). Detectable FIG. 1 or FIG. 2 polynucleotides include, for example, a FIG. 1 or FIG. 2 gene or fragment thereof, a FIG. 1 or FIG. 2 mRNA, alternative splice variants, FIG. 1 or FIG. 2 mRNAs, and recombinant DNA or RNA molecules containing a FIG. 1 or FIG. 2 polynucleotide.

The expression profile of each gene of FIG. 1 or FIG. 2 makes it a diagnostic marker for local and/or metastasized disease, and provides information on the growth or oncogenic potential of a biological sample. In particular, the status of a gene of FIG. 1 or FIG. 2-related protein provides information useful for predicting susceptibility to particular disease stages, progression, and/or tumor aggressiveness. The invention provides methods and assays for determining the expression or mutational status of a gene of FIG. 1 or FIG. 2 and diagnosing cancers that express a gene of FIG. 1 or FIG. 2, such as cancers of the tissues listed in Table I. For example, because each gene of FIG. 1 or FIG. 2 mRNA is highly expressed in cancers relative to normal tissue, assays that evaluate the levels of FIG. 1 or FIG. 2 mRNA transcripts or proteins in a biological sample are used to diagnose a disease associated with dysregulation of a gene set forth in FIG. 1 or FIG. 2, and can provide prognostic information useful in defining appropriate therapeutic options.

The expression status of the genes set forth in FIG. 1 or FIG. 2 provides information including the presence, stage and location of dysplastic, precancerous and cancerous cells, predicting susceptibility to various stages of disease, and/or for gauging tumor aggressiveness. Moreover, the expression profile makes it useful as an imaging reagent for metastasized disease. Consequently, an aspect of the invention is directed to the various molecular prognostic and diagnostic methods for examining the status of these genes and proteins in biological samples such as those from individuals suffering from, or suspected of suffering from a pathology characterized by dysregulated cellular growth, such as cancer.

As described above, the status of the genes in FIG. 1 or FIG. 2 in a biological sample can be examined by a number of well-known procedures in the art. For example, the status of the genes in FIG. 1 or FIG. 2 in a biological sample taken from a specific location in the body can be examined by evaluating the sample for the presence or absence of a gene of FIG. 1 or FIG. 2-related protein expressing cells (e.g. those that express FIG. 1 or FIG. 2 mRNAs or proteins). This examination can provide evidence of dysregulated cellular growth, for example, when gene of FIG. 1 or FIG. 2-related protein-expressing cells are found in a biological sample that does not normally contain such cells (such as a lymph node), because such alterations in the status of the genes in FIG. 1 or FIG. 2 in a biological sample are often associated with dysregulated cellular growth. Specifically, one indicator of dysregulated cellular growth is the metastases of cancer cells from an organ of origin (such as the prostate) to a different area of the body (such as a lymph node). In this context, evidence of dysregulated cellular growth is important for example because occult lymph node metastases can be detected in a substantial proportion of patients with prostate cancer, and such metastases are associated with known predictors of disease progression (see, e.g., Murphy et al., Prostate 42(4): 315-317 (2000);Su et al, Semin. Surg. Oncol. 18(1): 17-28 (2000) and Freeman et al., J Urol August 1995 154(2 Pt 1):474-8).

In one aspect, the invention provides methods for monitoring gene of FIG. 1 or FIG. 2 expression by determining the status of FIG. 1 or FIG. 2 gene products expressed by cells from an individual suspected of having a disease associated with dysregulated cell growth (such as hyperplasia or cancer) and then comparing the status so determined to the status of FIG. 1 or FIG. 2 gene products in a corresponding normal sample. The presence of aberrant FIG. 1 or FIG. 2 gene products in the test sample relative to the normal sample provides an indication of the presence of dysregulated cell growth within the cells of the individual.

In another aspect, the invention provides assays useful in determining the presence of cancer in an individual, comprising detecting a significant increase in FIG. 1 or FIG. 2 mRNA or protein expression in a test cell or tissue sample relative to expression levels in the corresponding normal cell or tissue. The presence of FIG. 1 or FIG. 2 mRNA can, for example, be evaluated in tissue samples including but not limited to those listed in Table I. The presence of significant gene of FIG. 1 or FIG. 2-related protein expression or over-expression in any of these tissues is useful to. indicate the emergence, presence and/or severity of a cancer, where the corresponding normal tissues do not express FIG. 1 or FIG. 2 mRNA or express it at lower levels.

In a related embodiment, the status of a gene of FIG. 1 or FIG. 2 is determined at the protein level rather than at the nucleic acid level. For example, such a method comprises determining the level of a gene of FIG. 1 or FIG. 2-related protein expressed by cells in a test tissue sample and comparing the level so determined to the level of a gene of FIG. 1 or FIG. 2-related protein expressed in a corresponding normal sample. In one embodiment, the presence of a gene of FIG. 1 or FIG. 2-related protein is evaluated, for example, using immunohistochemical methods. Antibodies of the invention or binding partners capable of detecting a gene of FIG. 1 or FIG. 2-related protein expression are used in a variety of assay formats well known in the art for this purpose.

In a further embodiment, one can evaluate the status of a FIG. 1 or FIG. 2 nucleotide in a biological sample in order to identify perturbations in the structure of these molecules. These perturbations can include insertions, deletions, substitutions and the like. Such evaluations are useful because perturbations in the nucleotide and amino acid sequences are observed in a large number of proteins associated with a growth dysregulated phenotype (see, e.g., Marrogi et al., 1999, J. Cutan. Pathol. 26(8):369-378). For example, a mutation in the sequence of a FIG. 1 or FIG. 2 gene can indicate the presence or promotion of a tumor. Such assays therefore have diagnostic and predictive value where a mutation in a FIG. 1 or FIG. 2 gene indicates a potential loss of function or increase in tumor growth.

A wide variety of assays for observing perturbations in nucleotide and amino acid sequences are well known in the art. For example, the size and structure of nucleic acid sequences of a gene of FIG. 1 or FIG. 2, or the gene products of one of these genes are observed by the Northern, Southern, Western, PCR and DNA sequencing protocols as discussed herein. In addition, other methods for observing perturbations in nucleotide and amino acid sequences such as single strand conformation polymorphism analysis are well known in the art (see, e.g., U.S. Pat. Nos. 5,382,510 issued Sep. 7, 1999, and 5,952,170 issued Jan. 17, 1995).

Additionally, one can examine the methylation status of a FIG. 1 or FIG. 2 gene in a biological sample. Aberrant demethylation and/or hypermethylation of CpG islands in gene 5′ regulatory regions frequently occurs in immortalized and transformed cells, and can result in altered expression of various genes. For example, promoter hypermethylation of the pi-class glutathione S-transferase (a protein expressed in normal prostate but not expressed in >90% of prostate carcinomas) appears to permanently silence transcription of this gene and is the most frequently detected genomic alteration in prostate carcinomas (De Marzo et al, Am. J. Pathol. 155(6): 1985-1992 (1999)). In addition, this alteration is present in at least 70% of cases of high-grade prostatic intraepithelial neoplasia (PIN) (Brooks et al, Cancer Epidemiol. Biomarkers Prev., 1998, 7:531-536). In another example, expression of the LAGE-I tumor specific gene (which is not expressed in normal prostate but is expressed in 25-50% of prostate cancers) is induced by deoxy-azacytidine in lymphoblastoid cells, suggesting that tumoral expression is due to demethylation (Lethe et al., Int. J. Cancer 76(6): 903-908 (1998)). A variety of assays for examining methylation status of a gene are well known in the art. For example, one can utilize, in Southern hybridization approaches, methylation-sensitive restriction enzymes that cannot cleave sequences that contain methylated CpG sites to assess the methylation status of CpG islands. In addition, MSP (methylation specific PCR) can rapidly profile the methylation status of all the CpG sites present in a CpG island of a given gene. This procedure involves initial modification of DNA by sodium bisulfite (which will convert all unmethylated cytosines to uracil) followed by amplification using primers specific for methylated versus unmethylated DNA. Protocols involving methylation interference can also be found for example in Current Protocols In Molecular Biology, Unit 12, Frederick M. Ausubel et al eds., 1995.

Gene amplification is an additional method for assessing the status of a FIG. 1 or FIG. 2 gene. Gene amplification is measured in a sample directly, for example, by conventional Southern blotting or Northern blotting to quantitate the transcription of mRNA (Thomas, 1980, Proc. Natl. Acad. Sci. USA, 77:5201-5205), dot blotting (DNA analysis), or in situ hybridization, using an appropriately labeled probe, based on the sequences provided herein. Alternatively, antibodies are employed that recognize specific duplexes, including DNA duplexes, RNA duplexes, and DNA-RNA hybrid duplexes or DNA-protein duplexes. The antibodies in turn are labeled and the assay carried out where the duplex is bound to a surface, so that upon the formation of duplex on the surface, the presence of antibody bound to the duplex can be detected.

Biopsied tissue or peripheral blood can be conveniently assayed for the presence of cancer cells using for example, Northern, dot blot or RT-PCR analysis to detect expression of a gene of FIG. 1 or FIG. 2. The presence of RT-PCR amplifiable FIG. 1 or FIG. 2 mRNA provides an indication of the presence of cancer. RT-PCR assays are well known in the art. RT-PCR detection assays for tumor cells in peripheral blood are currently being evaluated for use in the diagnosis and management of a number of human solid tumors. In the prostate cancer field, these include RT-PCR assays for the detection of cells expressing PSA and PSM (Verkaik et al., 1997, Urol. Res. 25:373-384; Ghossein et al., 1995, J. Clin. Oncol. 13:1195-2000; Heston et al, 1995, Clin. Chem 41:1687-1688).

A further aspect of the invention is an assessment of the susceptibility that an individual has for developing cancer. In one embodiment, a method for predicting susceptibility to cancer comprises detecting FIG. 1 or FIG. 2 mRNA or a gene of FIG. 1 or FIG. 2-related protein in a tissue sample, its presence indicating susceptibility to cancer, wherein the degree of FIG. 1 or FIG. 2 mRNA expression correlates to the degree of susceptibility. In a specific embodiment, the presence of a gene of FIG. 1 or FIG. 2-related protein in, e.g., prostate tissue is examined, with the presence of a gene of FIG. 1 or FIG. 2-related protein in the sample providing an indication of prostate cancer susceptibility (or the emergence or existence of a prostate tumor). Similarly, one can evaluate the integrity a gene of FIG. 1 or FIG. 2 nucleotide and amino acid sequences in a biological sample, in order to identify perturbations in the structure of these molecules such as insertions, deletions, substitutions and the like. The presence of one or more perturbations in genes or gene products of the invention in the sample is an indication of cancer susceptibility (or the emergence or existence of a tumor).

The invention also comprises methods for gauging tumor aggressiveness. In one embodiment, a method for gauging aggressiveness of a tumor comprises determining the level of FIG. 1 or FIG. 2 mRNA or a gene of FIG. 1 or FIG. 2-related protein expressed by tumor cells, comparing the level so determined to the level of FIG. 1 or FIG. 2 mRNA or a gene of FIG. 1 or FIG. 2-related protein expressed in a corresponding normal tissue taken from the same individual or a normal tissue reference sample, wherein the degree of FIG. 1 or FIG. 2 mRNA or a gene of FIG. 1 or FIG. 2-related protein expression in the tumor sample relative to the normal sample indicates the degree of aggressiveness. In a specific embodiment, aggressiveness of a tumor is evaluated by determining the extent to which a gene of FIG. 1 or FIG. 2 is expressed in the tumor cells, with higher expression levels indicating more aggressive tumors. Another embodiment is the evaluation of the integrity of FIG. 1 or FIG. 2 nucleotide sequences in a biological sample, in order to identify perturbations in the structure of these molecules such as insertions, deletions, substitutions and the like. The presence of one or more perturbations indicates more aggressive tumors.

Another embodiment of the invention is directed to methods for observing the progression of a malignancy in an individual over time. In one embodiment, methods for observing the progression of a malignancy in an individual over time comprise determining the level of FIG. 1 or FIG. 2 mRNA or a gene of FIG. 1 or FIG. 2-related protein expressed by cells in a sample of the tumor, comparing the level so determined to the level of FIG. 1 or FIG. 2 mRNA or a gene of FIG. 1 or FIG. 2-related protein expressed in an equivalent tissue sample taken from the same individual at a different time, wherein the degree of FIG. 1 or FIG. 2 mRNA or a gene of FIG. 1 or FIG. 2-related protein expression in the tumor sample over time provides information on the progression of the cancer. In a specific embodiment, the progression of a cancer is evaluated by determining FIG. 1 or FIG. 2 gene or protein expression in the tumor cells over time, where increased expression over time indicates a progression of the cancer. Also, one can evaluate the integrity of FIG. 1 or FIG. 2 nucleotide sequences in a biological sample in order to identify perturbations in the structure of these molecules such as insertions, deletions, substitutions and the like, where the presence of one or more perturbations indicates a progression of the cancer.

The above diagnostic approaches can be combined with any one of a wide variety of prognostic and diagnostic protocols known in the art. For example, another embodiment of the invention is directed to methods for observing a coincidence between the expression of a FIG. 1 or FIG. 2 gene and/or FIG. 1 or FIG. 2 gene products (or perturbations in a FIG. 1 or FIG. 2 gene and/or FIG. 1 or FIG. 2 gene products) and a factor that is associated with malignancy, as a means for diagnosing and prognosticating the status of a tissue sample. A wide variety of factors associated with malignancy can be utilized, such as the expression of genes associated with malignancy (e.g. PSA, PSCA and PSM expression for prostate cancer, etc.) as well as gross cytological observations (see, e.g., Bocking et al., 1984, Anal. Quant. Cytol. 6(2):74-88; Epstein, 1995, Hum. Pathol. 26(2):223-9; Thorson et al, 1998, Mod. Pathol. 11(6):543-51; Baisden et al, 1999, Am. J. Surg. Pathol. 23(8):918-24). Methods for observing a coincidence between the expression of a FIG. 1 or FIG. 2 gene and/or FIG. 1 or FIG. 2 gene products (or perturbations in a FIG. 1 or FIG. 2 gene and/or FIG. 1 or FIG. 2 gene products) and another factor that is associated with malignancy are useful, for example, because the presence of a set of specific factors that coincide with disease provides information crucial for diagnosing and prognosticating the status of a tissue sample.

In one embodiment, methods for observing a coincidence between the expression of a FIG. 1 or FIG. 2 gene and FIG. 1 or FIG. 2 gene products (or perturbations in a FIG. 1 or FIG. 2 gene and/or FIG. 1 or FIG. 2 gene products) and another factor associated with malignancy entails detecting the overexpression of FIG. 1 or FIG. 2 mRNA and/or protein in a tissue sample; detecting the overexpression of PSA mRNA or protein in a tissue sample (or PSCA or PSM expression, etc.), and observing a coincidence of FIG. 1 or FIG. 2 mRNA and/or protein and PSA mRNA or protein overexpression (or PSCA or PSM expression). In a specific embodiment, the expression of a gene of FIG. 1 or FIG. 2 and PSA mRNA in prostate tissue is examined, where the coincidence of a FIG. 1 or FIG. 2 gene and PSA mRNA overexpression in the sample indicates the existence of prostate cancer, prostate cancer susceptibility or the emergence or status of a prostate tumor.

Methods for detecting and quantifying the expression of FIG. 1 or FIG. 2 mRNA or protein are described herein, and standard nucleic acid and protein detection and quantification technologies are well known in the art. Standard methods for the detection and quantification of FIG. 1 or FIG. 2 mRNA include in situ hybridization using labeled FIG. 1 or FIG. 2 gene riboprobes, Northern blot and related techniques using FIG. 1 or FIG. 2 polynucleotide probes, RT-PCR analysis using primers specific for FIG. 1 or FIG. 2 genes, and other amplification type detection methods, such as, for example, branched DNA, SISBA, TMA and the like. In a specific embodiment, semi-quantitative RT-PCR is used to detect and quantify FIG. 1 or FIG. 2 mRNA expression. Any number of primers capable of amplifying a FIG. 1 or FIG. 2 gene can be used for this purpose, including but not limited to the various primer sets specifically described herein. In a specific embodiment, polyclonal or monoclonal antibodies specifically reactive with a wild-type gene of FIG. 1 or FIG. 2-related protein can be used in an immunohistochemical assay of biopsied tissue.

Diagnostic and Prognostic Embodiments of the Invention.

As disclosed herein, polynucleotides, polypeptides, reactive cytotoxic T cells (CTL), reactive helper T cells (HTL) and anti-polypeptide antibodies of the invention are used in well known diagnostic, prognostic and therapeutic assays that examine conditions associated with dysregulated cell growth such as cancer, in particular the cancers listed in Table I (see, e.g., both its specific pattern of tissue expression as well as its overexpression in certain cancers as described for example in Example 2).

Because metastases involves the movement of cancer cells from an organ of origin (such as the lung or prostate gland etc.) to a different area of the body (such as a lymph node), assays which examine a biological sample for the presence of cells expressing FIG. 1 or FIG. 2 polynucleotides and/or polypeptides can be used to provide evidence of metastasis. For example, when a biological sample from tissue that does not normally contain a gene of FIG. 1 or FIG. 2 or a gene of FIG. 1 or FIG. 2-related protein-expressing cells (e.g., a lymph node) is found to contain a gene of FIG. 1 or FIG. 2-related protein-expressing cells, this finding is indicative of metastasis.

Alternatively polynucleotides and/or polypeptides of the invention can be used to provide evidence of cancer, for example, when cells in a biological sample that do not normally express FIG. 1 or FIG. 2 genes or express FIG. 1 or FIG. 2 genes at a different level are found to express FIG. 1 or FIG. 2 genes or have an increased expression of FIG. 1 or FIG. 2 genes (see, e.g., the expression in the cancers of tissues listed in Table I and in patient samples etc. shown in the accompanying Figures). In such assays, artisans may further wish to generate supplementary evidence of metastasis by testing the biological sample for the presence of a second tissue restricted marker (in addition to a gene of FIG. 1 or FIG. 2-related protein) such as PSA, PSCA etc. (see, e.g., Alanen et al., Pathol. Res. Pract. 192(3): 233-237 (1996)).

Just as PSA polynucleotide fragments and polynucleotide variants are employed by skilled artisans for use in methods of monitoring PSA, a gene of FIG. 1 or FIG. 2 polynucleotide fragments and polynucleotide variants are used in an analogous manner. In particular, typical PSA polynucleotides used in methods of monitoring PSA are probes or primers which consist of fragments of the PSA cDNA sequence. Illustrating this, primers used to PCR amplify a PSA polynucleotide must include less than the whole PSA sequence to function in the polymerase chain reaction. In the context of such PCR reactions, skilled artisans generally create a variety of different polynucleotide fragments that can be used as primers in order to amplify different portions of a polynucleotide of interest or to optimize amplification reactions (see, e.g., Caetano-Anolles, G. Biotechniques 25(3): 472-476, 478-480 (1998); Robertson et al., Methods Mol. Biol. 98:121-154 (1998)). An additional illustration of the use of such fragments is provided in Example 4, where a gene of FIG. 1 or FIG. 2 polynucleotide fragments are used as a probe to show the expression of respective gene of FIG. 1 or FIG. 2 RNAs in cancer cells. In addition, variant polynucleotide sequences are typically used as primers and probes for the corresponding mRNAs in PCR and Northern analyses (see, e.g., Sawai et al, Fetal Diagn. Ther. Nov-Dec 11, 1996 (6):407-13 and Current Protocols In Molecular Biology, Volume 2, Unit 2, Frederick M. Ausubel et al. eds., 1995)). Polynucleotide fragments and variants are useful in this context where they are capable of binding to a target polynucleotide sequence (e.g., a FIG. 1 or FIG. 2 polynucleotide or variant thereof) under conditions of high stringency.

Furthermore, PSA polypeptides which contain an epitope that can be recognized by an antibody or T cell that specifically binds to that epitope are used in methods of monitoring PSA. Polypeptide fragments, polypeptide analogs or variants of a gene of FIG. 1 or FIG. 2-related protein can also be used in an analogous manner. This practice of using polypeptide fragments or polypeptide variants to generate antibodies (such as anti-PSA antibodies or T cells) is typical in the art with a wide variety of systems such as fusion proteins being used by practitioners (see, e.g., Current Protocols In Molecular Biology, Volume 2, Unit 16, Frederick M. Ausubel et al. eds., 1995).. In this context, each epitope(s) functions to provide the architecture with which an antibody or T cell is reactive. Typically, skilled artisans create a variety of different polypeptide fragments that can be. used in order to generate immune responses specific for different portions of a polypeptide of interest (see, e.g., U.S. Pat. No. 5,840,501 and U.S. Pat. No. 5,939,533). For example it may be preferable to utilize a polypeptide comprising one of the biological motifs of a gene of FIG. 1 or FIG. 2-related protein discussed herein or a motif-bearing subsequence which is readily identified by one of skill in the art based on motifs available in the art.

As shown herein, the FIG. 1 or FIG. 2 polynucleotides (as well as the FIG. 1 or FIG. 2 polynucleotide probes and anti-gene of FIG. 1 or FIG. 2-related protein antibodies or T cells used to identify the presence of these molecules) exhibit specific properties that make them useful in diagnosing cancers such as those listed in Table I. Diagnostic assays that measure the presence of gene of FIG. 1 or FIG. 2 gene products, in order to evaluate the presence or onset of a disease condition described herein, such as prostate cancer, are used to identify patients for preventive measures or further monitoring, as has been done so successfully with PSA. Moreover, these materials satisfy a need in the art for molecules having similar or complementary characteristics to PSA in situations where, for example, a definite diagnosis of metastasis of prostatic origin cannot be made on the basis of a test for PSA alone (see, e.g., Alanen et al., Pathol. Res. Pract. 192(3): 233-237 (1996)), and consequently, materials such as FIG. 1 or FIG. 2 polynucleotides and polypeptides (as well as the gene of FIG. 1 or FIG. 2 polynucleotide probes and anti-proteins of FIG. 1 or FIG. 2 antibodies used to identify the presence of these molecules) need to be employed to confirm a metastases of prostatic origin.

Finally, in addition to their use in diagnostic assays, the FIG. 1 or FIG. 2 polynucleotides disclosed herein have a number of other utilities such as their use in the identification of oncogenetic associated chromosomal abnormalities in the chromosomal region to which a FIG. 1 or FIG. 2 genes map (see Example 3 below). Moreover, in addition to their use in diagnostic assays, the gene of FIG. 1 or FIG. 2-related proteins and polynucleotides disclosed herein have other utilities such as their use in the forensic analysis of tissues of unknown origin (see, e.g., Takahama K Forensic Sci Int Jun 28, 1996 ;80(1-2): 63-9).

Additionally, gene of FIG. 1 or FIG. 2-related proteins or polynucleotides of the invention can be used to treat a pathologic condition characterized by the over-expression of gene of FIG. 1 or FIG. 2-related proteins. Antibodies or other molecules that react with proteins of the invention can be used to modulate the function of this molecule, and thereby provide a therapeutic benefit.

Inhibition of Transcription or Translation in Accordance with the Invention

The present invention also comprises various methods and compositions for inhibiting the transcription of a FIG. 1 or FIG. 2 gene. Similarly, the invention also provides methods and compositions for inhibiting the translation of the genes in FIG. 1 or FIG. 2-related mRNA into protein.

In one approach, a method of inhibiting the transcription of a FIG. 1 or FIG. 2 gene comprises contacting the FIG. 1 or FIG. 2 gene with a respective FIG. 1. or FIG. 2 antisense polynucleotide. In another approach, a method of inhibiting gene of FIG. 1 or FIG. 2-related mRNA translation comprises contacting a gene of FIG. 1 or FIG. 2-related mRNA with an antisense polynucleotide. In another approach, a gene of FIG. 1 or FIG. 2 specific ribozyme is used to cleave a gene of FIG. 1 or FIG. 2-related message, thereby inhibiting translation. Such antisense and ribozyme based methods can also be directed to the regulatory regions of a FIG. 1 or FIG. 2 gene, such as a promoter and/or enhancer element for a gene of FIG. 1 or FIG. 2. Similarly, proteins capable of inhibiting agene of FIG. 1 or FIG. 2 transcription factor are used to inhibit the gene of FIG. 1 or FIG. 2 mRNA transcription. The various polynucleotides and compositions useful in the aforementioned methods have been described above. The use of antisense and ribozyme molecules to inhibit transcription and translation is well known in the art.

Other factors that inhibit the transcription of a FIG. 1 or FIG. 2 gene by interfering with that gene's transcriptional activation are also useful to treat cancers expressing genes of FIG. 1 or FIG. 2.. Similarly, factors that interfere with a gene of FIG. 1 or FIG. 2 gene processing are useful to treat cancers that express genes of FIG. 1 or FIG. 2. Cancer treatment methods utilizing such factors are also within the scope of the invention.

General Considerations for Therapeutic Strategies

Gene transfer and gene therapy technologies can be used to deliver therapeutic polynucleotide molecules to tumor cells synthesizing a gene of FIG. 1 or FIG. 2-related protein (e.g., antisense, ribozyme, polynucleotides encoding intrabodies and other gene of FIG. 1 or FIG. 2-related inhibitory molecules). A number of gene therapy approaches are known in the art. Recombinant vectors encoding FIG. 1 or FIG. 2 antisense polynucleotides, ribozymes, factors capable of interfering with transcription of a gene of FIG. 1 or FIG. 2, and so forth, can be delivered to target tumor cells using such gene therapy approaches.

The above therapeutic approaches can be combined with any one of a wide variety of surgical, chemotherapy or radiation therapy regimens. The therapeutic approaches of the invention can enable the use of reduced dosages of chemotherapy (or other therapies) and/or less frequent administration, an advantage for all patients and particularly for those that do not tolerate the toxicity of the chemotherapeutic agent well.

The anti-tumor activity of a particular composition (e.g., antisense, ribozyme, intrabody), or a combination of such compositions, can be evaluated using various in vitro and in vivo assay systems. In vitro assays that evaluate therapeutic activity include cell growth assays, soft agar assays and other assays indicative of tumor promoting activity, binding assays capable of determining the extent to which a therapeutic composition will inhibit the binding of a gene of FIG. 1 or FIG. 2-related protein to one or more of its binding partners, etc.

In vivo, the effects of a therapeutic composition of the invention can be evaluated in a suitable animal model. For example, xenogenic prostate cancer models can be used, wherein human prostate cancer explants or passaged xenograft tissues are introduced into immune compromised animals, such as nude or SCID mice (Klein et al., 1997, Nature Medicine 3: 402-408). For example, PCT Patent Application WO 098/16628 and U.S. Pat. No. 6,107,540 describe various xenograft models of human prostate cancer capable of recapitulating the development of primary tumors, micrometastasis, and the formation of osteoblastic metastases characteristic of late stage disease. Efficacy can be predicted using assays that measure inhibition of tumor formation, tumor regression or metastasis, and the like.

In vivo assays that evaluate the promotion of apoptosis are useful in evaluating therapeutic compositions. In one embodiment, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980).

Therapeutic formulations can be solubilized and administered via any route capable of delivering the therapeutic composition to the tumor site. Potentially effective routes of administration include, but are not limited to, intravenous, parenteral, intraperitoneal, intramuscular, intratumor, intradermal, intraorgan, orthotopic, and the like. A preferred formulation for intravenous injection comprises the therapeutic composition in a solution of preserved bacteriostatic water, sterile unpreserved water, and/or diluted in polyvinylchloride or polyethylene bags containing 0.9% sterile Sodium Chloride for Injection, USP. Therapeutic protein preparations can be lyophilized and stored as sterile powders, preferably under vacuum, and then reconstituted in bacteriostatic water (containing for example, benzyl alcohol preservative) or in sterile water prior to injection.

Dosages and administration protocols for the treatment of cancers using the foregoing methods will vary with the method and the target cancer, and will generally depend on a number of other factors appreciated in the art.

Kits

For use in the diagnostic and therapeutic applications described herein, kits are also within the scope of the invention. Such kits can comprise a carrier, package or container that is compartmentalized to receive one or more containers such as vials, tubes, and the like, each of the container(s) comprising one of the separate elements to be used in the method. For example, the container(s) can comprise a probe that is or can be detectably labeled. Such probe can be an antibody or polynucleotide specific for a gene of FIG. 1 or FIG. 2-related protein or a FIG. 1 or FIG. 2 gene or message, respectively. Where the method utilizes nucleic acid hybridization to detect the target nucleic acid, the kit can also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label. The kit can include all or part of the nucleic acid sequences in a gene of FIG. 1 or FIG. 2 or analogs thereof, or a nucleic acid molecules that encodes such amino acid sequences.

The kit of the invention will typically comprise the container described above and one or more other containers comprising materials desirable from a commercial and user standpoint, including buffers, diluents, filters, needles, syringes; carrier, package, container, vial and/or tube labels listing contents and/or instructions for use, and package inserts with instructions for use.

A label can be present on the container to indicate that the composition is used for a specific therapy or non-therapeutic application, and can also indicate directions for either in vivo or in vitro use, such as those described above. Directions and or other information can also be included on an insert which is included with the kit.

### EXAMPLES

Various aspects of the invention are further described and illustrated by way of the several examples that follow, none of which are intended to limit the scope of the invention.

### Example 1

### SSH-Generated Isolation of a cDNA Fragment of the Invention

The suppression subtractive hybridization (SSH) cDNA fragments shown in FIG. 1 were derived from many different subtractions utilizing LAPC xenografts in differing states of androgen dependence and/ or castration as well as using cancer patient derived tissues. The cancer patient tissue SSHs utilized prostate, bladder, and kidney with tumors representing all stages and grades of the diseases. Information for additional sequences disclosed in the genes of FIG. 2 was derived from other clones and the use of various sequence databases.

Materials and Methods

LAPC Xenografts and Human Tissues

LAPC xenografts were obtained from Dr. Charles Sawyers (UCLA) and generated as described (Klein et al, 1997, Nature Med. 3: 402-408; Craft et al., 1999, Cancer Res.. 59: 5030-5036). Androgen dependent and independent LAPC xenografts were grown in male SCID mice and were passaged as small tissue chunks in recipient males. LAPC xenografts were derived from LAPC tumors. To generate the androgen independent (AI) xenografts, male mice bearing androgen dependent (AD) tumors were castrated and maintained for 2-3 months. After the tumors re-grew, the tumors were harvested and passaged in castrated males or in female SCID mice. Tissues from prostate, bladder, kidney, colon, lung, pancreas, ovary, and breast cancer patients as well as the corresponding normal tissues were stored frozen at −70 C. prior to RNA isolation.

RNA Isolation

Tumor tissue and cell lines were homogenized in Trizol reagent (Life Technologies, Gibco BRL) using 10 ml/ g tissue or 10 ml/ 10⁸ cells to isolate total RNA. Poly A RNA was purified from total RNA using Qiagen's Oligotex mRNA Mini and Midi kits. Total and mRNA were quantified by spectrophotometric analysis (O. D. 260/280 nm) and analyzed by gel electrophoresis.

Oligonucleotides

The following HPLC purified oligonucleotides were used.

DPNCDN (cDNA synthesis primer):

(SEQ ID NO:XX)

5′TTTTGATCAAGCTT₃₀3′
Adaptor 1:

(SEQ ID NO:XX)

5′CTAATACGACTCACTATAGGGCTCGAGCGGCCGCCCGGGCAG3′

(SEQ ID NO:XX)

3′GGCCCGTCCTAG5′
Adaptor 2:

(SEQ ID NO:XX)

5′GTAATACGACTCACTATAGGGCAGCGTGGTCGCGGCCGAG3′

(SEQ ID NO:XX)

3′CGGCTCCTAG5′
PCR primer 1:

(SEQ ID NO:XX)

5′CTAATACGACTCACTATAGGGC3′
Nested primer (NP)1:

(SEQ ID NO:XX)

5′TCGAGGGGCCGCCCGGGCAGGA3′
Nested primer (NP)2:

(SEQ ID NO:XX)

5′AGCGTGGTCGCGGCCGAGGA3′

Suppression Subtractive Hybridization

Suppression Subtractive Hybridization (SSH) was used to identify cDNAs corresponding to genes that are differentially expressed in cancer. The SSH reaction utilized cDNA from the prostate cancer xenografts, LAPC-4 AD, LAPC-4 AI, LAPC-9 AD, and LAPC-9AI as well as from prostate, bladder, and kidney cancer patients. Specifically, to isolate genes that are involved in the progression of androgen dependent (AD) prostate cancer to androgen independent (AI) cancer, experiments were conducted with the LAPC-9 AD and LAPC-4 AD xenograft in male SCID mice. Mice that harbored these xenografts were castrated when the tumors reached a size of 1 cm in diameter. The tumors regressed in size and temporarily stopped producing the androgen dependent protein PSA. Seven to fourteen days post-castration, PSA levels were detectable again in the blood of the mice. Eventually the tumors develop an Al phenotype and start growing again in the castrated males. Tumors were harvested at different time points after castration to identify genes that are turned on or off during the transition to androgen independence.

The cDNAs derived from LAPC-4 AD and LAPC-9 AD tumors (post-castration) were used as the source of the “tester” cDNAs, while the cDNAs from LAPC4-AD and LAPC-9 AD tumors (grown in intact male mouse) were used as the source of the “driver” cDNAs respectively. Some SSHs also used any combination of the LAPC-4 AD, LAPC-4 AI, LAPC-9AD, and LAPC9-AI xenografts as “tester” or “driver”. In addition, cDNAs derived from patient tumors of prostate, bladder and kidney cancer were used as “tester” while cDNAs derived from normal prostate, bladder, and kidney were used as “driver” respectively. Double stranded cDNAs corresponding to tester and driver cDNAs were synthesized from 2 μg of poly(A)+RNA isolated from the relevant xenograft tissue, as described above, using CLONTECH's PCR-Select cDNA Subtraction Kit and 1 ng of oligonucleotide DPNCDN as primer. First-strand and second-strand synthesis were carried out as described in the Kit's user manual protocol (CLONTECH Protocol No. PT1117-1, Catalog No. K1804-1). The resulting cDNA was digested with Dpn II for 3 hrs at 37° C. Digested cDNA was extracted with phenol/chloroform (1:1) and ethanol precipitated.

Tester cDNA was generated by diluting 1 μl of Dpn II digested cDNA from the relevant xenograft source (see above) (400 ng) in 5 μl of water. The diluted cDNA (2 μl , 160 ng) was then ligated to 2 μl of Adaptor 1 and Adaptor 2 (10 μM), in separate ligation reactions, in a total volume of 10 μl at 16° C. overnight, using 400 u of T4 DNA ligase (CLONTECH). Ligation was terminated with 1 μl of 0.2 M EDTA and heating at 72° C. for 5 min.

The first hybridization was performed by adding 1.5 μl (600 ng) of driver cDNA to each of two tubes containing 1.5 μl (20 ng) Adaptor 1- and Adaptor 2- ligated tester cDNA. In a final volume of 4 pi, the samples were overlaid with mineral oil, denatured in an MJ Research thermal cycler at 98° C. for 1.5 minutes, and then were allowed to hybridize for 8 hrs at 68° C. The two hybridizations were then mixed together with an additional 1 μl of fresh denatured driver cDNA and were allowed to hybridize overnight at 68° C. The second hybridization was then diluted in 200 μl of 20 mM Hepes, pH 8.3, 50 mM NaCl, 0.2 mM EDTA, heated at 70° C. for 7 min. and stored at −20° C.

PCR Amplification, Cloning and Sequencing of Gene Fragments Generated from SSH:

To amplify gene fragments resulting from SSH reactions, two PCR amplifications were performed. In the primary PCR reaction 1 μl of the diluted final hybridization mix was added to 1 μ l of PCR primer 1 (10 μM), 0.5 μl dNTP mix (10 μM), 2.5 μl 10 × reaction buffer (CLONTECH) and 0.5 μl 50 × Advantage cDNA polymerase Mix (CLONTECH) in a final volume of 25 μl. PCR 1 was conducted using the following conditions: 75° C. for 5 min., 94° C. for 25 sec., then 27 cycles of 94° C. for 10 sec, 66° C. for 30 sec, 72° C. for 1.5 min. Five separate primary PCR reactions were performed for each experiment. The products were pooled and diluted 1:10 with water. For the secondary PCR reaction, 1 μl from the pooled and diluted primary PCR reaction was added to the same reaction mix as used for PCR 1, except that primers NP1 and NP2 (10 μM) were used instead of PCR primer 1. PCR 2 was performed using 10- 12 cycles of 94° C. for 10 sec, 68° C. for 30 sec, and 72oC. for 1.5 minutes. The PCR products were analyzed using 2% agarose gel electrophoresis.

The PCR products were inserted into pCR2.1 using the T/A vector cloning kit (Invitrogen). Transformed E. coli were subjected to blue/white and ampicillin selection. White colonies were picked and arrayed into 96 well plates and were grown in liquid culture overnight. To identify inserts, PCR amplification was performed on 1 μl of bacterial culture using the conditions of PCR1 and NP1 and NP2 as primers. PCR products were analyzed using 2% agarose gel electrophoresis.

Bacterial clones were stored in 20% glycerol in a 96 well format. Plasmid DNA was prepared, sequenced, and subjected to nucleic acid homology searches of the GenBark, dBest, and NCI-CGAP databases.

A full-length cDNA clone can be identified by assembling EST fragments homologous to the SSH fragment into a large contiguous sequence with an ORF and amplifying the ORF by PCR using xenograft, prostate, bladder, kidney, prostate cancer, bladder cancer, or kidney cancer first strand cDNA.

### Example 2

### Chromosomal Mapping

Chromosomal localization can implicate genes in disease pathogenesis. Several chromosome mapping approaches are available including fluorescent in situ hybridization (FISH), human/hamster radiation hybrid (RH) panels (Walter et al., 1994; Nature Genetics 7:22; Research Genetics, Huntsville Ala.), human-rodent somatic cell hybrid panels such as is available from the Coriell Institute (Camden, N.J.), and genomic viewers utilizing BLAST homologies to sequenced and mapped genomic clones (NCBI, Bethesda, Md.).

Using FIG. 2 gene sequences and the NCBI BLAST tool: (see World Wide Web URL www.ncbi.nhn.nih.gov/genome/seq/page.cgi?F=HsBlast.html&&ORG=Hs), placed the genes of FIG. 1 and FIG. 2 to the chromosome locations listed in Table II.

Accordingly, as the human genes set forth in FIG. 2 map to the designated chromosomes, polynucleotides encoding different regions of a gene of FIG. 1 or FIG. 2-related can be used to characterize cytogenetic abnormalities on a respective chromosome. For example, when chromosomal abnormalities in a chromosome listed in Table XXII have been identified as frequent cytogenetic abnormalities in different cancers (see, e.g., Lai et al., 2000, Clin. Cancer Res. 6(8):3172-6; Oya and Schulz, 2000, Br. J. Cancer 83(5):626-3 1; Svaren et al., Sep. 12, 2000, J. Biol. Chem.); polynucleotides encoding specific regions of a gene of FIG. 1 or FIG. 2-related protein provide new tools that are used to delineate, with greater precision than previously possible, the specific nature of the cytogenetic abnormalities in this region of the respective chromosome that contribute to the malignant phenotype. In this context, these polynucleotides satisfy a need in the art for expanding the sensitivity of chromosomal screening in order to identify more subtle and less common chromosomal abnormalities (see, e.g., Evans et al., 1994, Am. J. Obstet. Gynecol. 171(4):1055-1057).

### Example 3

### Expression Analysis of a Nucleic Acid of the Invention in Normal Tissues and Patient Specimens

Expression analysis by RT-PCR and Northern analysis demonstrated that normal tissue expression of a gene of FIG. 1 or FIG. 2 is restricted predominantly to the tissues set forth in Table I.

Therapeutic applications for a gene of FIG. 1 and FIG. 2 include use as a small molecule therapy and/or a vaccine (T cell or antibody) target. Diagnostic applications for a gene of FIG. 1 or FIG. 2 include use as a diagnostic marker for local and/or metastasized disease. The restricted expression of a gene of FIG. 2 in normal tissues makes it useful as a tumor target for diagnosis and therapy. Expression analysis of a gene of FIG. 1 or FIG. 2 provides information useful for predicting susceptibility to advanced stage disease, rate of progression, and/or tumor aggressiveness. Expression status of a gene of FIG. 1 or FIG. 2 in patient samples, tissue arrays and/or cell lines may be analyzed by: (i) immunohistochemical analysis; (ii) in situ hybridization; (iii) RT-PCR analysis on laser capture micro-dissected samples; (iv) Western blot analysis; and (v) Northern analysis.

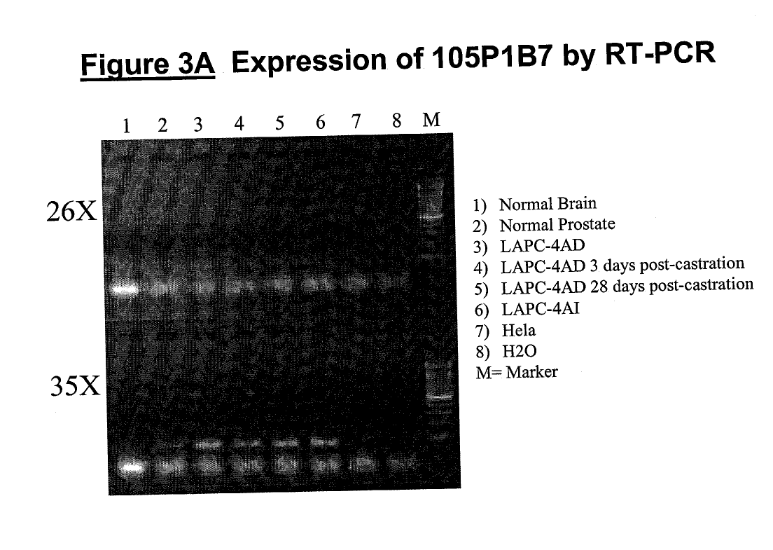

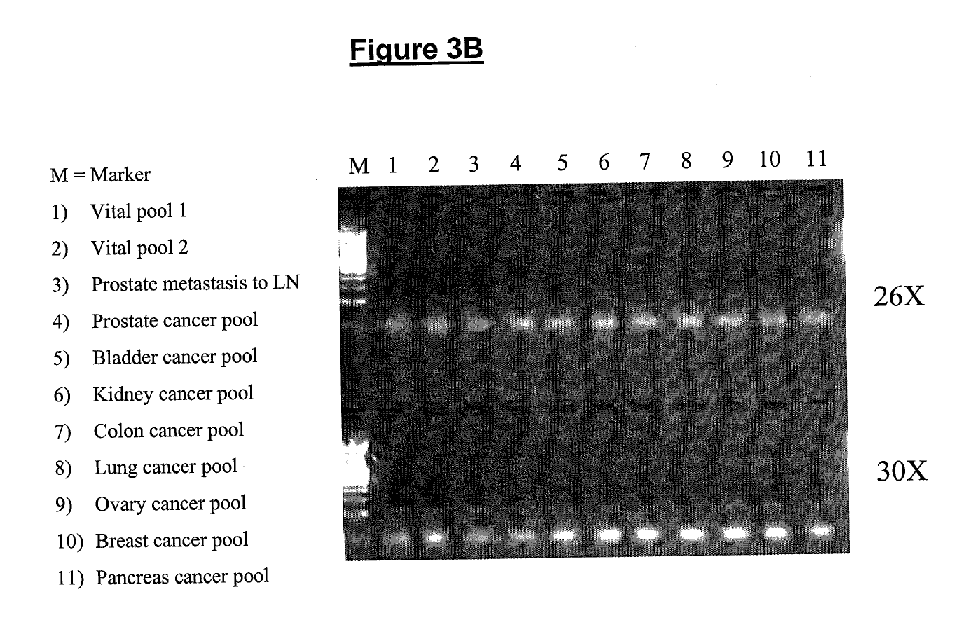

RT-PCR analysis and Northern blotting were used to evaluate gene expression in a selection of normal and cancerous urological tissues. The results are summarized in FIGS. 3-20.

RT-PCR Expression Analysis

First strand cDNAs can be generated from 1 μg of mRNA with oligo (dT) 12-18 priming using the Gibco-BRL Superscript Preamplification system. The manufacturer's protocol was used which included an incubation for 50 min at 42° C. with reverse transcriptase followed by RNAse H treatment at 37° C. for 20 min. After completing the reaction, the volume can be increased to 200 μl with water prior to normalization. First strand cDNAs from 16 different normal human tissues can be obtained from Clontech.

Normalization of the first strand cDNAs from multiple tissues was performed by using the primers 5′ atatcgccgcgctcgtcgtcgacaa3′ (SEQ ID NO: XX) and 5′ agccacacgcagctcattgtagaagg 3′ (SEQ ID NO: XX) to amplify β-actin. First strand cDNA (5 μl) were amplified in a total volume of 50 μl containing 0.4 μM primers, 0.2 μM each dNTPs, 1XPCR buffer (Clontech, 10 mM Tris-HCL, 1.5 mM MgCl₂, 50 mM KCl, pH8.3) and 1× Klentaq DNA polymerase (Clontech). Five μl of the PCR reaction can be removed at 18, 20, and 22 cycles and used for agarose gel electrophoresis. PCR was performed using an MJ Research thermal cycler under the following conditions: Initial denaturation can be at 94° C. for 1 minute 15 seconds, followed by 18, 20, and 26 cycles (where a cycle is 94° C. for 45 seconds, 58° C. for 45 seconds, 72° C. for 45 seconds) then finally, 72° C. for 5 minutes. A final extension at 72° C. was carried out for 2 min. After agarose gel electrophoresis, the band intensities of the 283 b.p. β-actin bands from multiple tissues were compared by visual inspection. Dilution factors for the first strand cDNAs were calculated to result in equal β-actin band intensities in all tissues after 22 cycles of PCR. Three rounds of normalization can be required to achieve equal band intensities in all tissues after 22 cycles of PCR.

To determine expression levels of the gene, 5 μl of normalized first strand cDNA are analyzed by PCR using 26, and 30 cycles of amplification. Semi-quantitative expression analysis can be achieved by comparing the PCR products at cycle numbers that give light band intensities. RT-PCR expression analysis is performed on first strand cDNAs generated using pools of tissues from multiple samples. The cDNA normalization was demonstrated in every experiment using beta-actin PCR.

Northern Blot Expression Analysis

Expression of mRNA in normal and cancerous human tissues was analyzed by northern blotting. Expression in normal tissues was analyzed using two multiple tissue blots (Clontech; Palo Alto, Calif.), comprising a total of 16 different normal human tissues, using labeled SSH fragment as a probe. To further analyze expression in prostate cancer tissues, northern blotting was performed on RNA derived from the LAPC xenografts and/or prostate cancer patient samples. In addition, expression in other cancers was studied using patient samples and/or various cancer cell lines.

FIG. 3 shows expression of 105P1B7 by RT-PCR. (A) First strand cDNA was prepared from normal brain, normal prostate, LAPC-4AD, LAPC-4AD at 3 and 28 days after castration, LAPC-4AI, and Hela cancer cell lines. Normalization was performed by PCR using primers to actin and GAPDH. Semi-quantitative PCR, using primers to 105P1B7, was performed at 26 and 35 cycles of amplification. Results show expression of 105P1B7 in normal prostate and in the LAPC prostate cancer xenografts, but not in normal brain nor in the Hela cell line. (BY First strand cDNA was prepared from vital pool 1 (liver, lung and kidney), vital pool 2 (pancreas, colon and stomach), prostate metastasis to lymph node (LN), prostate cancer pool, bladder cancer pool kidney cancer pool, colon cancer pool, lung cancer pool, ovary cancer pool, breast cancer pool, and pancreas cancer pool. Expression of 105P1B7 was detected in all cancer pools tested and in the vital pools.

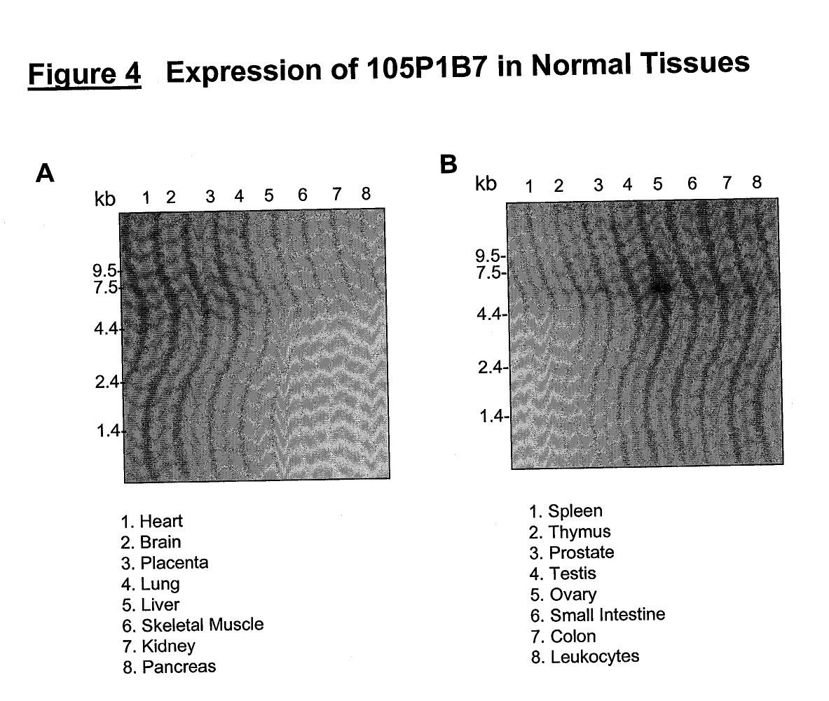

FIG. 4 shows expression of 105P1B7 in normal tissues. Two multiple tissue northern blots (Clontech) both with 2 μg of mRNA/lane, were probed with the 105P1B7 SSH fragment. Size standards in kilobases (kb) are indicated on the side. Results show expression of approximately 6.5 kb 105P1B7 transcript in ovary and weakly in normal prostate, but not in the other normal tissues tested.

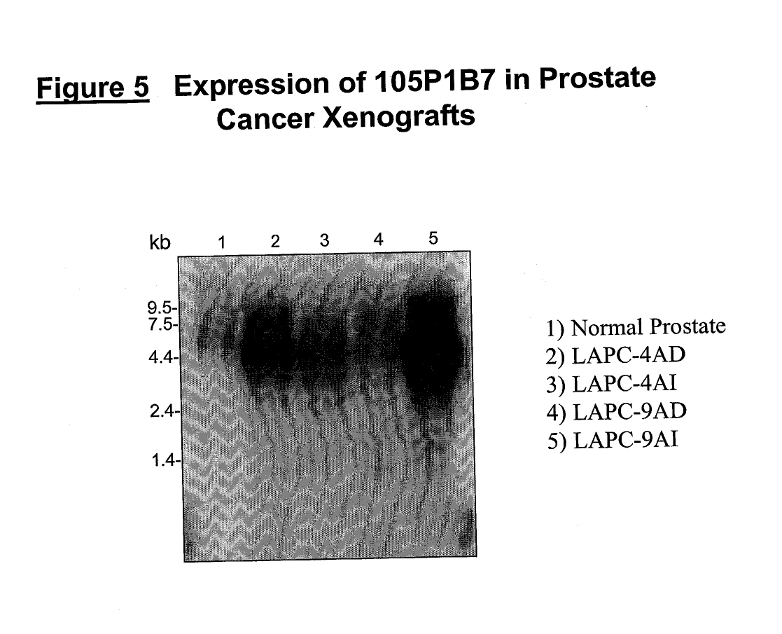

FIG. 5 shows expression of 105P1B7 in prostate cancer xenografts. RNA was extracted from normal prostate (NP), LAPC prostate cancer xenografts, LAPC-4AD, LAPC-4AI, LAPC-9AD and LAPC-9AI. Northern blot with 10 mg of total RNA/lane was probed with 105P1B7 SSH sequence. Size standards in kilobases (kb) are indicated on the side. The results show strong expression of 105P1B7 in all xenografts tissues and in normal prostate.

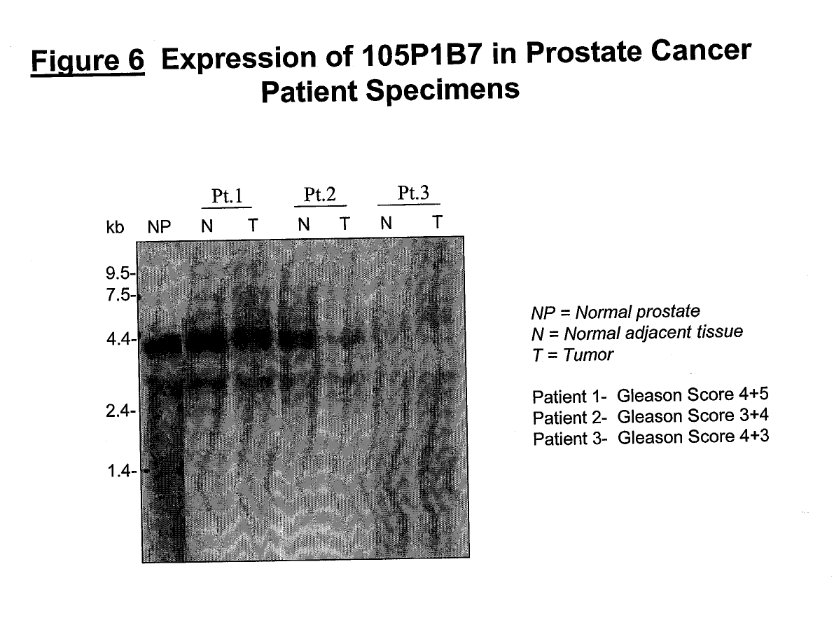

FIG. 6 shows expression of 105P1B7 in prostate cancer patient specimens. RNA was extracted from normal prostate (NP), prostate cancer patient tumors (T) and their normal adjacent tissues (N). Northern blot with 10 mg of total RNA/lane was probed with 105P1B7 SSH sequence. Size standards in kilobases (kb) are indicated on the side. The results show strong expression of 105P1B7 in normal prostate and in patient prostate cancer specimens.

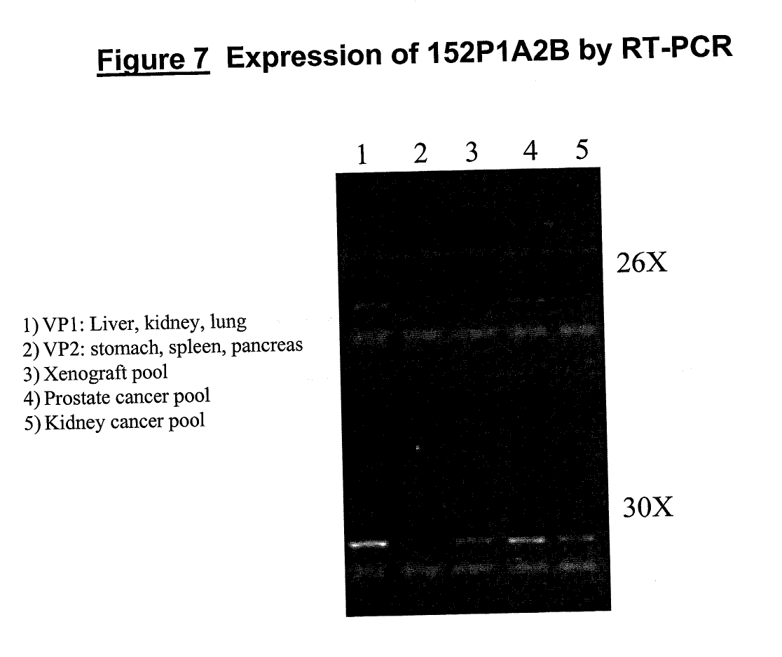

FIG. 7 shows expression of 152P1A2B by RT-PCR. First strand cDNA was prepared from vital pool 1 (liver, lung and kidney), vital pool 2 (pancreas, colon and stomach), LAPC prostate cancer xenograft pool (LAPC-4AD, LAPC-4AI, LAPC-9AD and LAPC-9AI), prostate cancer pool, and kidney cancer pool. Normalization was performed by PCR using primers to actin and GAPDH. Semi-quantitative PCR, using primers to 152P1A2B, was performed at 26 and 30 cycles of amplification. Results show strong expression of 83P4B8 in xenograft pool, prostate cancer pool, and kidney cancer pool. Expression was detected in the vital pool 1 but not in vital pool 2.

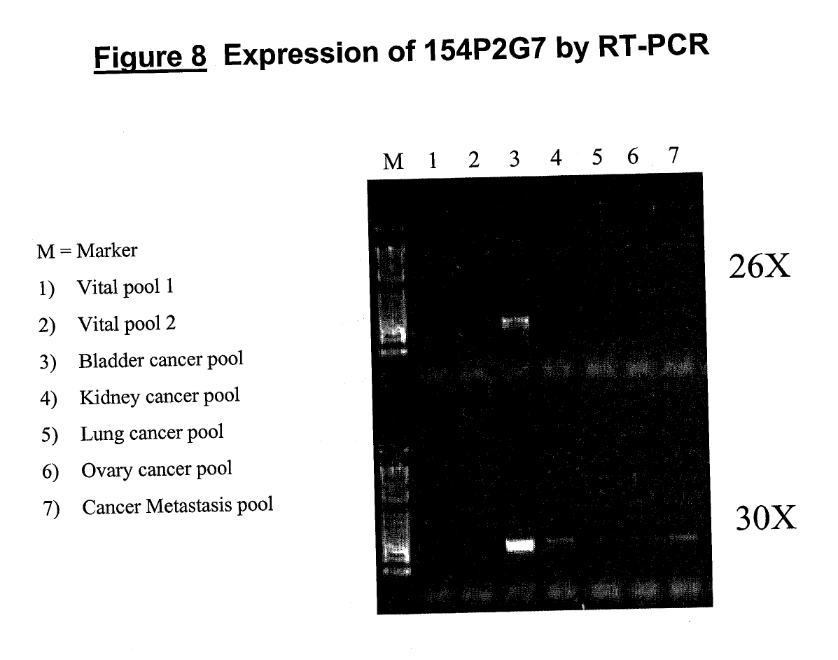

FIG. 8 shows expression of 154P2G7 by RT-PCR. First strand cDNA was prepared from vital pool 1 (liver, lung and kidney), vital pool 2 (pancreas, colon and stomach), bladder cancer pool, kidney cancer pool, lung cancer pool, ovary cancer pool, and cancer metastasis pool. Normalization was performed by PCR using primers to actin and GAPDH. Semi-quantitative PCR, using primers to 154P2G7, was performed at 26 and 30 cycles of amplification. Results show strong expression of 154P2G7 in bladder cancer pool. Expression was also detected in kidney cancer pool, lung cancer pool, ovary cancer pool and cancer metastasis pool, but not in the two vital pools tested.

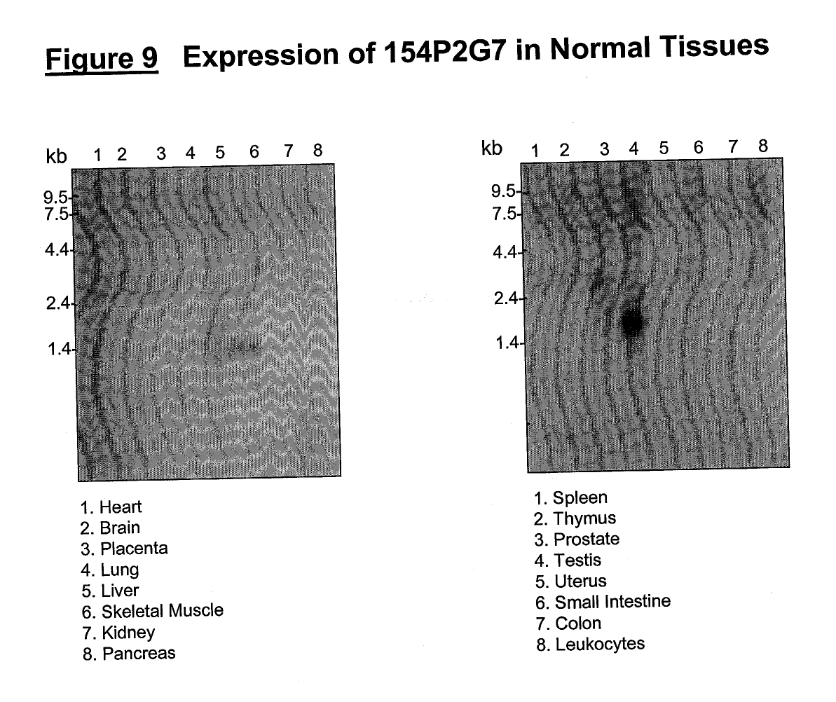

FIG. 9 shows expression of 154P2G7 in normal tissues. Two multiple tissue northern blots (Clontech), both with 2 μg of mRNA/lane, were probed with the 154P2G7 SSH fragment. Size standards in kilobases (kb) are indicated on the side. Results show expression of an approximately 1.8 kb 154P 2G7 transcript in testis. Very low expression was also detected in skeletal muscle and brain, but not in the other normal tissues tested.

FIG. 10 shows expression of 154P2G7 in bladder cancer patient specimens. RNA was extracted from bladder cancer cell lines (CL; UM-UC-3, SCaBER), normal bladder (Nb), and bladder cancer patient tumors (T). Northern blots with 10 μg of total RNA were probed with the 154P2G7 SSH sequence. Size standards in kilobases are indicated on the side. Results show expression of 154P2G7 in patient bladder cancer tissues, but not in normal bladder, nor in the bladder cancer cell lines tested.

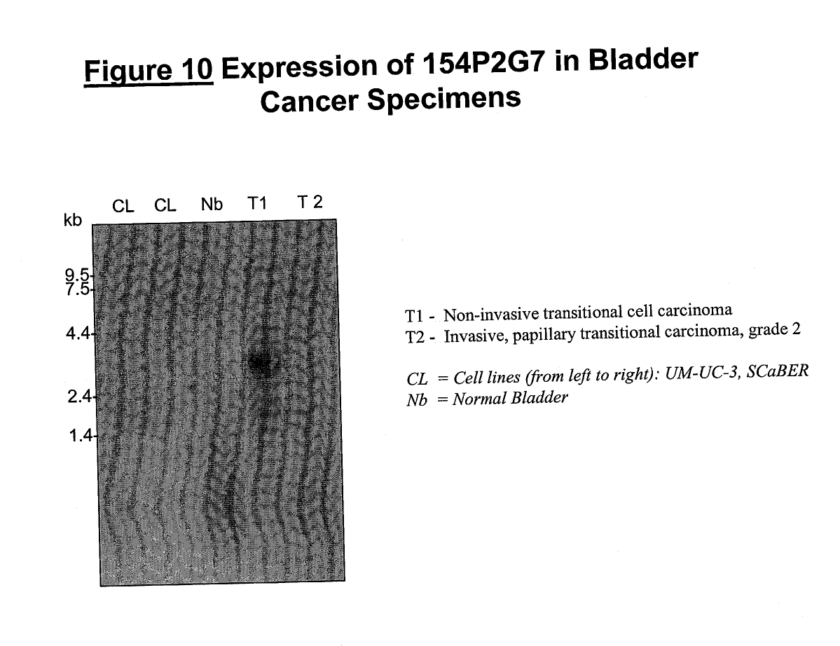

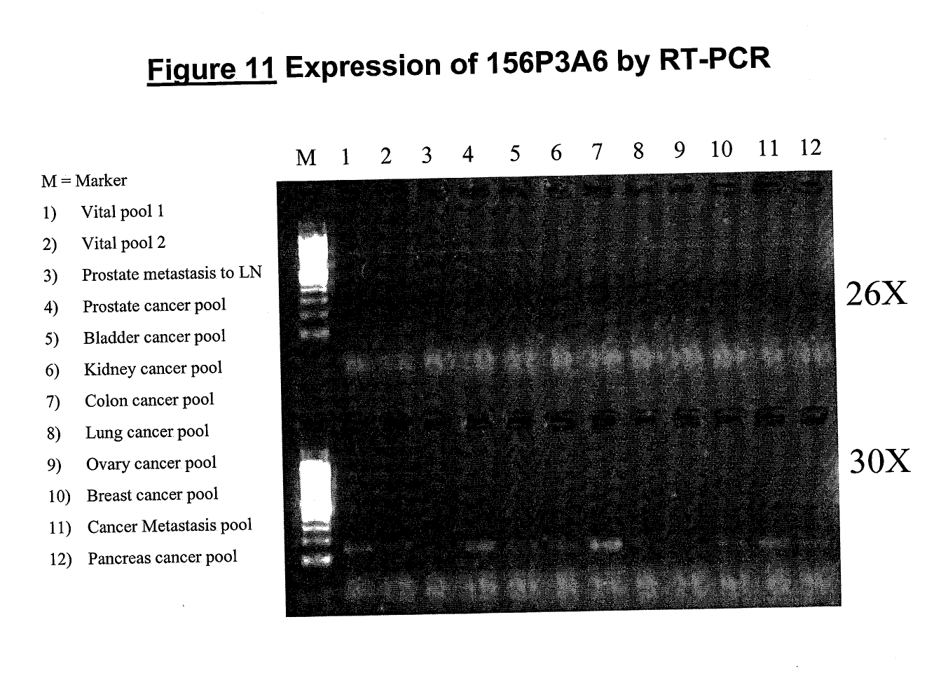

FIG. 11 shows expression of 156P3A6 by RT-PCR. First strand cDNA was prepared from vital pool 1 (VP1: liver, lung and kidney), vital pool 2 (VP2, pancreas, colon and stomach), prostate metastasis to lymph node (LN), prostate cancer pool, bladder cancer pool, kidney cancer pool, colon cancer pool, lung cancer pool, ovary cancer pool, breast cancer pool, cancer metastasis pool, and pancreas cancer pool. Normalization was performed by PCR using primers to actin and GAPDH. Semi-quantitative PCR, using primers to 156P 3A6, was performed at 26 and 30 cycles of amplification. Results show strong expression of 156P3A 6 in prostate cancer pool, colon cancer pool, and cancer metastasis pool. Expression was also detected in the other cancer pools tested and in the vital pools.

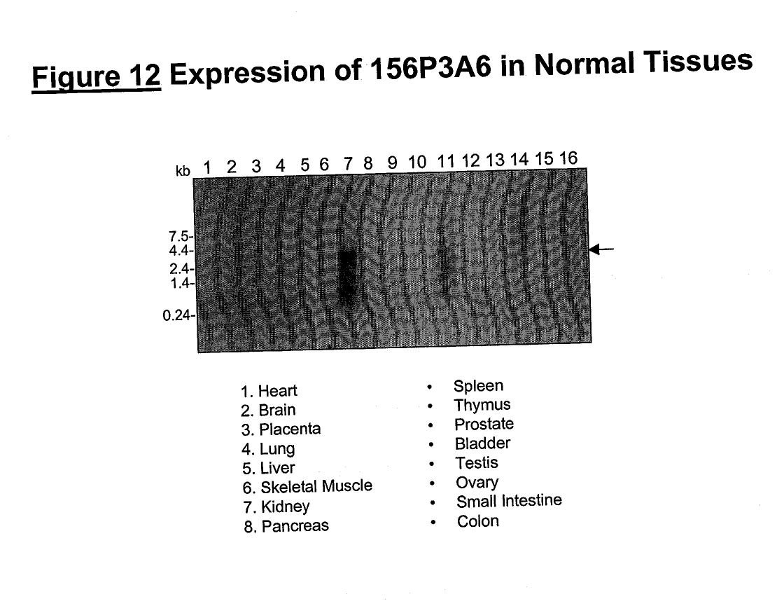

FIG. 12 shows expression of 156P3A6 in normal tissues. Multiple tissue northern blot, with 10 mg of total RNA/lane, was probed with the 156P3A6 SSH fragment. Size standards in kilobases (kb) are indicated on the side. The results show exclusive expression of an approximately 3.0 kb 156P3A6 transcript in kidney and prostate.

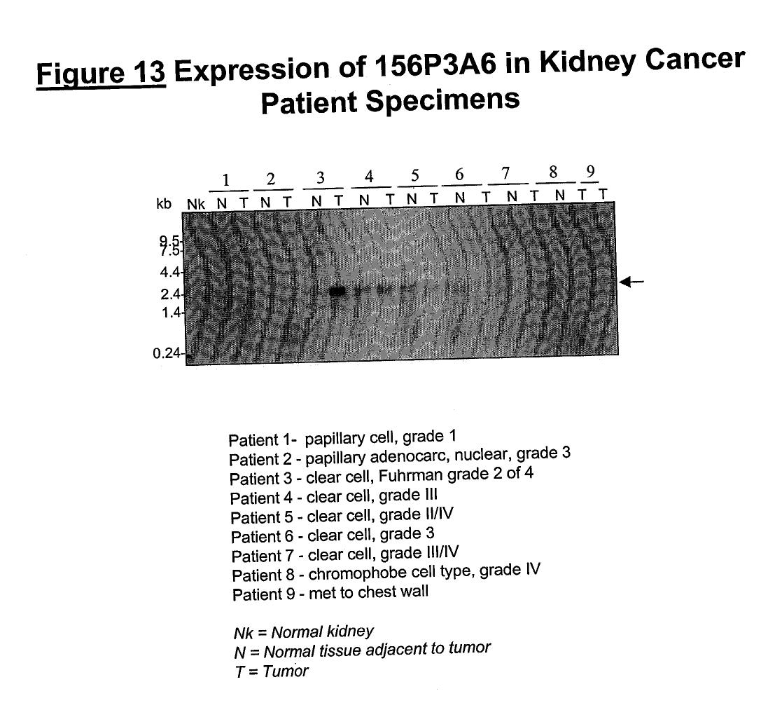

FIG. 13 shows expression of 156P3A6 in kidney cancer patient specimens. RNA was extracted from normal kidney (Nk), kidney tumors (T) and their normal adjacent tissues (N) derived from kidney cancer patients. Northern blots with 10 mg of total RNA/lane were probed with the 156P3A 6 SSH fragment. Size standards in kilobases (kb) are indicated on the side. The results show expression of 156P3A6 in kidney tumors and their normal adjacent tissues. Expression detected in kidney tumors is stronger than expression detected in normal kidney.

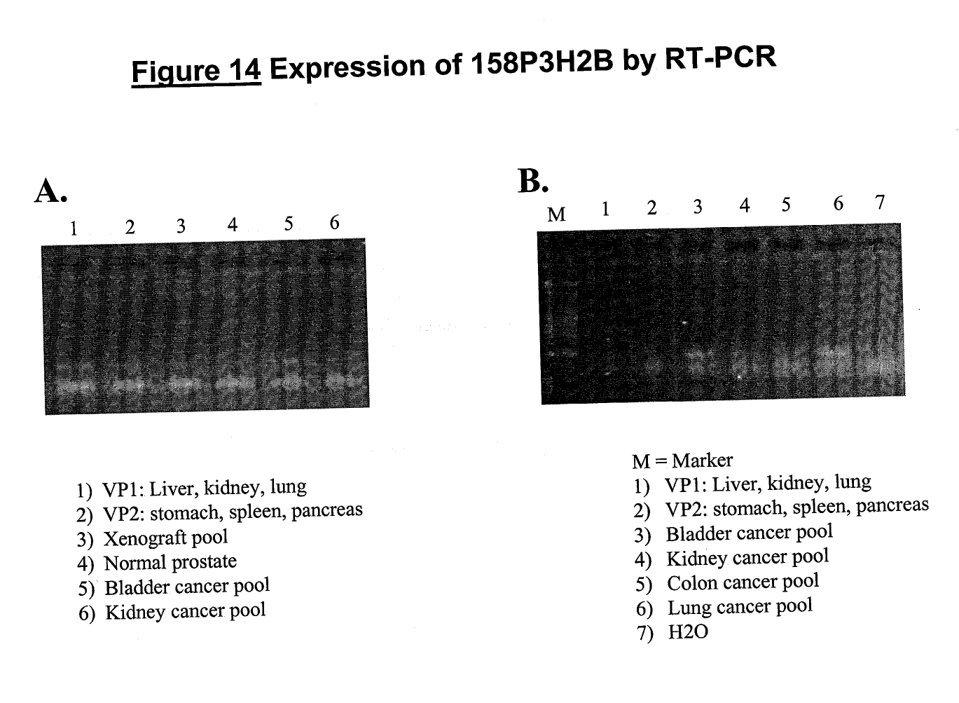

FIG. 14 shows expression of 158P3H2B by RT-PCR. First strand cDNA was prepared (A) from vital pool 1 (VP 1: liver, lung and kidney), vital pool 2 (VP2, pancreas, spleen and stomach), LAPC xenograft pool (LAPC-4AD, LAPC-4AI, LAPC-9AD and LAPC-9AI), normal prostate, bladder cancer pool, and kidney cancer pool; (B) from vital pool 1 (VP1: liver, lung and kidney), vital pool 2 (VP2, pancreas, spleen and stomach), bladder cancer pool, kidney cancer pool, colon cancer pool and lung cancer pool. Normalization was performed by PCR using primers to actin and GAPDH. Semi-quantitative PCR, using primers to 158P3H2B, was performed at 30 cycles of amplification. Results show expression of 158P3H2B in bladder cancer pool, kidney cancer pool, colon cancer pool, and lung cancer pool but not in the normal tissues tested.

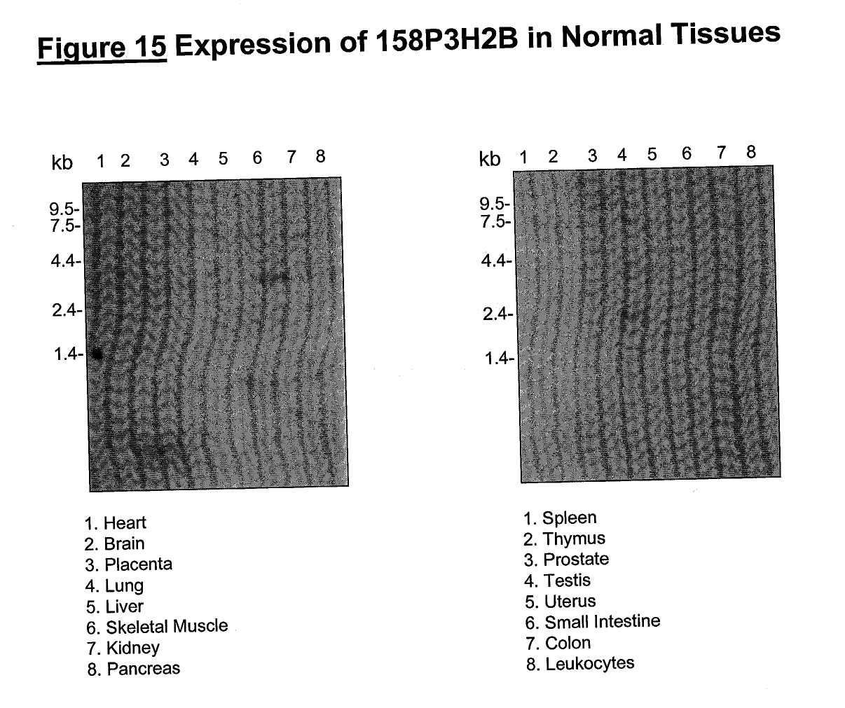

FIG. 15 shows expression of 158P3H2B in normal human tissues. Two multiple tissue northern blots (Clontech) both with 2 μg of mRNA/lane, were probed with the 158P3H 2B SSH fragment. Size standards in kilobases (kb) are indicated on the side. The results show exclusive expression of a 2.4 kb 158P3H2B transcript in testis but not in the other tissues tested.

FIG. 16 shows expression of 158P3H2B in bladder cancer patient samples. RNA was extracted from bladder cancer cell lines (CL: UM-UC-3, J82, SCABER), normal bladder (Nb), bladder tumors (T) and their normal adjacent tissues (N) harvested from bladder cancer patients. Northern blots with 10 mg of total RNA/lane were probed with the 158P3H2B SSH fragment. Size standards in kilobases (kb) are indicated on the side. The results show strong expression of 158P3H2B in all 5 bladder tumors tested and in one normal adjacent tissue, but not in normal bladder. Also strong expression was seen in the two cell lines, UM-UC-3 and SCABER, and to much lower level in J82.

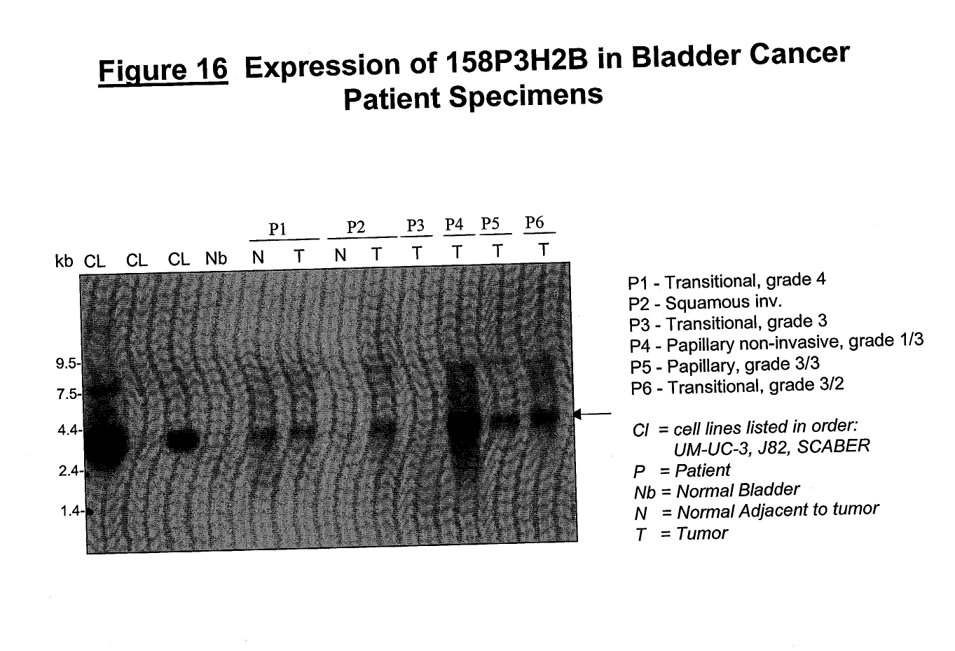

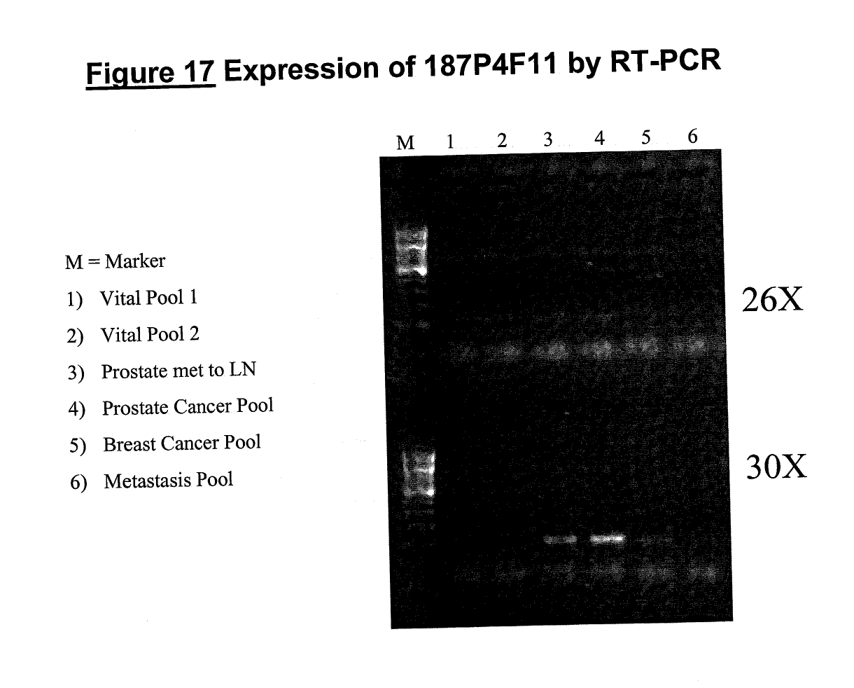

FIG. 17 shows expression of 187P4Fl 1 by RT-PCR. First strand cDNA was prepared from vital pool 1 (liver, lung and kidney), vital pool 2 (pancreas, colon and stomach), prostate metastasis to lymph node (LN), prostate cancer pool, breast cancer pool, and cancer metastasis pool. Normalization was performed by PCR using primers to actin and GAPDH. Semi-quantitative PCR, using primers to 187P4F 11, was performed at 26 and 30 cycles of amplification. Results show strong expression of 187P4Fl 1 in prostate cancer pool, breast cancer pool, and cancer metastasis pool, but not in the vital pool. Expression of 187P4F11 was also detected in prostate metastasis to LN indicating the 187P4F11 can be a marker for cancer metastasis.

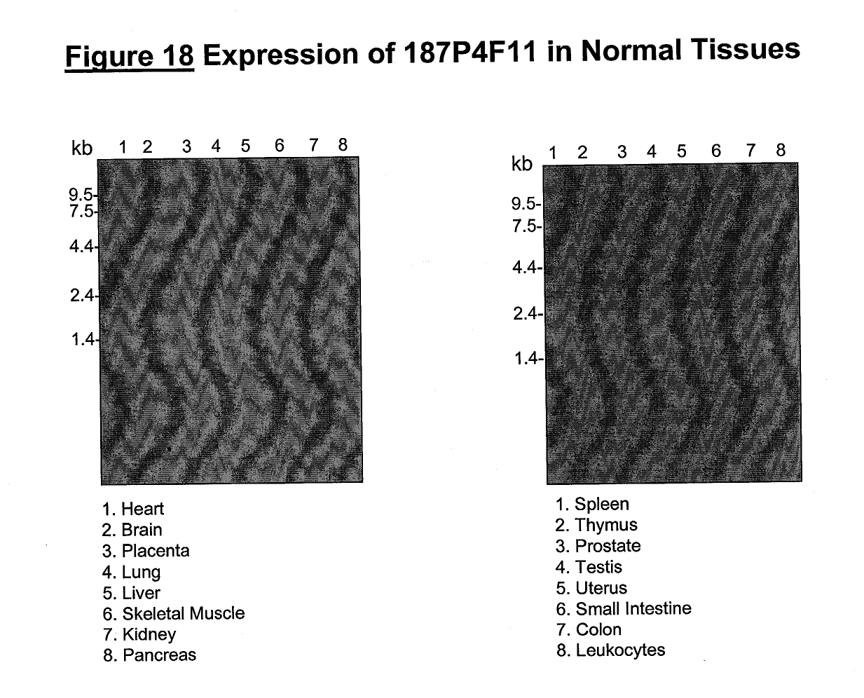

FIG. 18 shows expression of 187P4F11 in normal tissues. Two multiple tissue northern blots (Clontech), both with 2 μg of mRNA/lane, were probed with the 187P4F11 SSH fragment. Size standards in kilobases (kb) are indicated on the side. Results show absence of 187P4F11 in all 16 normal tissues tested.

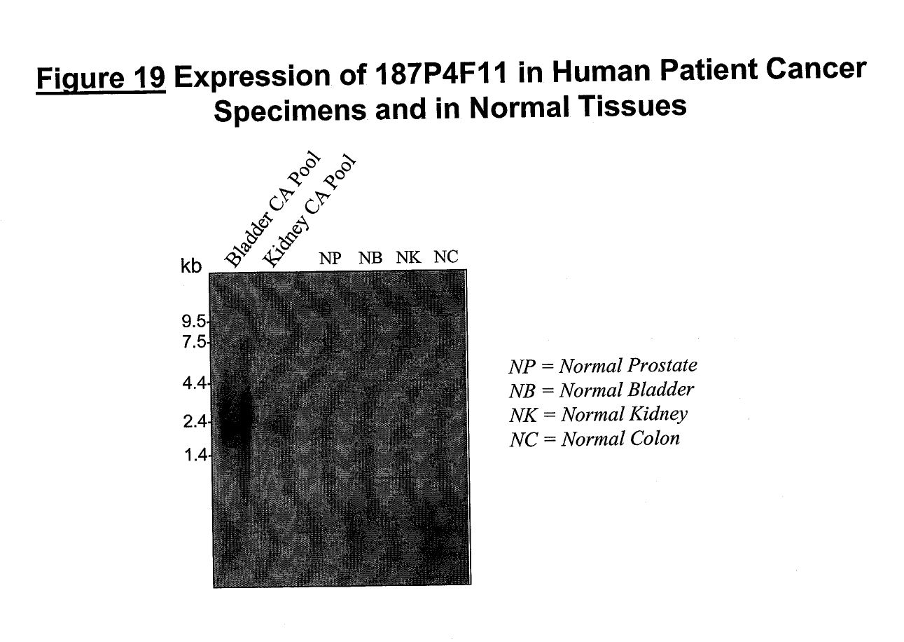

FIG. 19 shows expression of 187P4F11 in patient cancer specimens and normal tissues. RNA was extracted from a pool of three prostate cancers (PC), kidney cancers, as well as from normal prostate (NP), normal bladder (NB), normal kidney (NK), and normal colon (NC). Northern blot with 10 mg of total RNA/lane was probed with 187P4F11 SSH sequence. Size standards in kilobases (kb) are indicated on the side. Results show expression of 187P4F11 in the bladder cancers and kidney cancers, but not in the normal tissues tested.

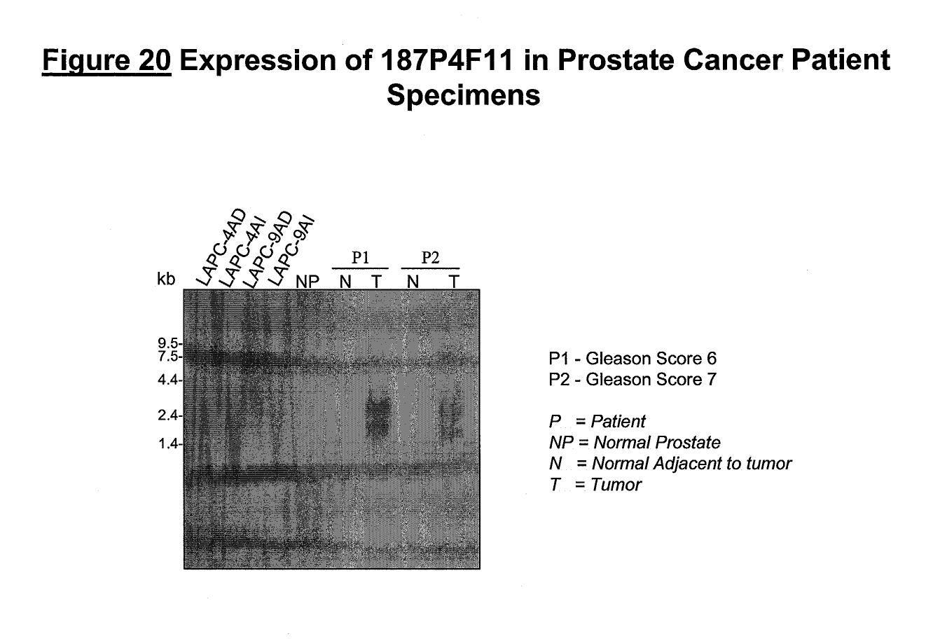

FIG. 20 shows expression of 187P4F11 in prostate cancer patient specimens. RNA was extracted from LAPC xenograft tissues, LAPC-4AD, LAPC-4AI, LAPC-9AD, LAPC-9AI, normal prostate (NP), prostate cancer patient tumors (T) and their normal adjacent tissues (N). Northern blot with 10 mg of total RNA/lane was probed with 83P4B8 SSH sequence. Size standards in kilobases (kb) are indicated on the side. The results show strong expression of approximately 2 and 2.8 kb 187P4F11 transcripts in the patient prostate cancer specimens, but not in normal prostate, nor in the xenograft tissues.

Throughout this application, various website data content, publications, patent applications and patents are referenced. (Websites are referenced by their Uniform Resource Locator, or URL, addresses on the World Wide Web.) The disclosures of each of these references are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

Tables

TABLE I
Exemplary Tissues that Express a Nucleic Acid Sequence
of FIG. 1 and/or FIG. 2 When Malignant.

| | Prostate | Bladder | Kidney | Colon | Lung | Ovary | Breast | Pancreas | Other |
|---|---|---|---|---|---|---|---|---|---|
| 105P1B7 | X | X | X | X | X | X | X | X | |
| 152P1A2B | X | | X | | | | | | |
| 154P2G7 | X | X | X | | X | X | | | |
| 156P3A6 | X | X | X | X | X | X | X | X | |
| 158P3H2B | | X | X | X | X | | | | |
| 187P4F11 | X | | X | | | | X | | |

TABLE II
Chromosomal localization of Nucleic Acid Sequences
from FIG. 1 and FIG. 2.

| | Chromosomal |
|---|---|
| Target | Localization |
| 105P1B7 | ND^{†} |
| 152P1A2B | 11q13.1 |
| 154P2G7 | 8q23 |
| 156P3A6 | 5p15.1 |
| 158P3H2B | 1q32.1 |
| 187P4F11 | 15 |

^{†}ND = Not determined

1. A composition comprising:
a substance that a) modulates the status of a nucleic acid sequence of FIG. 1 or FIG. 2 (SEQ ID NOS:______) , or b) a molecule that is modulated by a nucleic acid sequence of FIG. 1 or FIG. 2, whereby the status of a cell that expresses a nucleic acid sequence of FIG. 1 or FIG. 2 is modulated.

2. A composition of claim 1, wherein the molecule that is modulated by a respective nucleic acid sequence of FIG. 1 or FIG. 2 is RNA, where the RNA corresponds to the respective nucleic acid sequence of FIG. 1 or FIG. 2.

3. A composition of claim 1 wherein the cell that expresses a nucleic acid sequence of FIG. 1 or FIG. 2 is from a tissue set forth in Table I, respectively for the nucleic acid sequence of FIG. 1 or FIG. 2.

4. A composition of claim 1, further comprising a physiologically acceptable carrier.

5. A pharmaceutical composition that comprises the composition of claim 1 in a human unit dose form

6. A composition of claim 1 wherein the substance comprises a nucleic acid sequence of FIG. 1 or FIG. 2.

7. A composition of claim 1 wherein the substance comprises a nucleic acid sequence related to a nucleic acid sequence of FIG. 1 or FIG. 2.

8. A nucleic acid sequence of claim 7 that has at least 90% similarity, homology or identity to an entire amino acid sequence shown in FIG. 1 or FIG. 2 (SEQ ID NOS:______).

9. A nucleic acid sequence of claim 7 wherein T is substituted with U.

10. A nucleic acid sequence of claim 7 that further comprises a nucleotide sequence that encodes a protein.

11. A composition comprising a nucleic acid sequence that is fully complementary to a nucleic acid sequence of claim 6.

12. A composition comprising a nucleic acid sequence that is fully complementary to a nucleic acid sequence of claim 7.

13. A composition of claim 1 wherein the substance comprises:
a) a ribozyme that cleaves a nucleic acid sequence of FIG. 1 or FIG. 2; or,
b) a nucleic acid molecule that encodes the ribozyme.

14. A method of inhibiting growth of cancer cells that express a nucleic acid sequence of FIG. 1 or FIG. 2, the method comprising:
administering to the cells the composition of claim 1.

15. A method of claim 14 of inhibiting growth of cancer cells that express a nucleic acid sequence of FIG. 1 or FIG. 2, the method comprising steps of:
administering to said cells a nucleic acid sequence comprising a nucleic acid sequence of FIG. 1 or FIG. 2 or comprising a nucleic acid sequence complementary to a nucleic acid sequence of FIG. 1 or FIG. 2.

16. A method of claim 14 of inhibiting growth of cancer cells that express a nucleic acid sequence of FIG. 1 or FIG. 2, the method comprising steps of:
administering to said cells a ribozyme that cleaves the nucleic acid sequence of FIG. 1 or FIG. 2.

17. A method for detecting, in a sample, the presence of a nucleic acid sequence of FIG. 1 or FIG. 2, comprising steps of:
contacting the sample with a substance of claim 1 that specifically binds to the nucleic acid sequence of FIG. 1 or FIG. 2-related nucleic acid sequence; and,
determining that there is a complex of the substance with the substance with the FIG. 1 or FIG. 2-related nucleic acid sequence.

18. A method of claim 45 further comprising a step of: taking the sample from a patient who has or who is suspected of having a cancer.

19. A method of claim 17 for detecting the presence of a nucleic acid sequence of FIG. 1 or FIG. 2 mRNA in a sample comprising:
producing cDNA from mRNA in the sample by reverse transcription using at least one primer;
amplifying the cDNA so produced using a nucleic acid sequence of FIG. 1 or FIG. 2 nucleic acid sequences as sense and antisense primers, wherein the nucleic acid sequence of FIG. 1 or FIG. 2 used as the sense and antisense primers serve to amplify a nucleic acid sequence of FIG. 1 or FIG. 2 cDNA; and,
detecting the presence of the amplified nucleic acid sequence of FIG. 1 or FIG. 2 cDNA.

20. A method of claim 17 for monitoring expression of a nucleic acid sequence of FIG. 1 or FIG. 2 in a biological sample from an individual who has or who is suspected of having a cancer, the method comprising:
determining the status of a nucleotide of FIG. 1 or FIG. 2 expression by cells in a tissue sample from the individual;
comparing the status so determined to the status of the nucleotide of FIG. 1 or FIG. 2 expression in a corresponding normal sample; and,
identifying the presence of aberrant expression of the nucleotide of FIG. 1 or FIG. 2 in the sample relative to the normal sample.

21. The method of claim 20 further comprising a step of determining if there is an elevated gene product level from a nucleotide of FIG. 1 or FIG. 2, wherein the gene product is RNA or DNA or amino acid,
whereby the presence of elevated expression in the test sample relative to the normal tissue sample indicates the presence or status of a cancer.

22. A method of claim 21 wherein the cancer occurs in a tissue set forth in Table I.

23. A composition that comprises, consists essentially of, or consists of a nucleic acid sequence of FIG. 1 or FIG. 2.

24. A composition of claim 23 wherein T is substituted with U in the nucleic acid sequence.
